(19) United States
(12) Patent Application Publication
(10) Pub. No.: US 2005/0060102 A1
(43) Pub. Date: Mar. 17, 2005

(54) Interactive correlation of compound information and genomic information

(76) Inventors: David J. O'Reilly, Palo Alto, CA (US); Alan H. Roter, Redwood City, CA (US); Keith Bostian, Atherton, CA (US); David J. Morgans JR., Los Altos, CA (US)

Correspondence Address: HOWREY SIMON ARNOLD & WHITE, LLP, c/o IP DOCKETING DEPARTMENT, 2941 FAIRVIEW PARK DRIVE, SUITE 200, FALLS CHURCH, VA, 22042-2924, US

(21) Appl. No.: 10/854,609

(22) Filed: May 24, 2004

Related U.S. Application Data
(63) Continuation-in-part of application No. 09/977,064, filed on Oct. 11, 2001.
(60) Provisional application No. 60/240,118, filed on Oct. 12, 2000.

(51) Int. Cl. G06F 19/00; G01N 33/48; G01N 33/50

(52) U.S. Cl. 702/20

(57) ABSTRACT

An interactive system for facilitating hypothesis construction by correlating and presenting gene expression data, bioassay data, and compound activity data, and associating gene and compound function information with product information, and facilitating product purchase, is disclosed.

### RELATED APPLICATIONS

This application is a continuation-in-part of U.S. Ser. No. 09/977,064 filed Oct. 11, 2001 which claims priority from U.S. provisional application 60/240,118 filed Oct. 12, 2000, each of which is hereby incorporated by reference herein.

### FIELD OF THE INVENTION

This invention relates to methods and products for identifying pharmaceutical leads, correlating information regarding gene expression, biological assays and other relevant information, and facilitating the purchase of related products.

### BACKGROUND OF THE INVENTION

Genomic sequence information is now available for several organisms, and additional data is added continuously. However, only a small fraction of the open reading frames now sequenced correspond to genes of known function: the function of most polynucleotide sequences, and any encoded proteins, is still unknown. These genes are now studied by means of, inter alia, polynucleotide arrays, which quantify the amount of mRNA produced by a test cell (or organism) under specific conditions. “Chemical genomic annotation” is the process of determining the transcriptional and bioassay response of one or more genes to exposure to a particular chemical, and defining and interpreting such genes in terms of the classes of chemicals for which they interact. A comprehensive library of chemical genomic (also referred to herein as “chemogenomic”) annotations would enable one to design and optimize new pharmaceutical lead compounds based on the probable transcriptional and biomolecular profile of a hypothetical compound with certain characteristics. Additionally, one can use chemical genomic annotations to determine relationships between genes (for example, as members of a signal pathway or protein-protein interaction pair), and aid in determining the causes of side effects and the like. Finally, presenting the drug design researcher with a body of chemical genomic annotation information will generate research hypotheses that will stimulate follow-on experimental design, and therefor enable and stimulate purchase of related products to execute such experiments.

Sabatini et al., U.S. Pat. No. 5,966,712 disclosed a database and system for storing, comparing and analyzing genomic data.

Maslyn et al., U.S. Pat. No. 5,953,727 disclosed a relational database for storing genomic data.

Kohler et al., U.S. Pat. No. 5,523,208 disclosed a database and method for comparing polynucleotide sequences and the predicted functions of their encoded proteins.

Fujiyama et al., U.S. Pat. No. 5,706,498 disclosed database and retrieval system, for identifying genes of similar sequence.

### SUMMARY OF THE INVENTION

We have now invented a system and method for analyzing and exploring the data resulting from chemical genomic annotation experiments, and for facilitating the design by a user of further experiments related to the user's goals, and thereby encouraging the purchase by the user of products related to the data and additional experiments.

One aspect of the invention is a method for evaluating a test compound for biological activity, comprising: providing a database comprising a plurality of reference gene expression profiles, each profile comprising a representation of the expression level of a plurality of genes in a test cell exposed to a reference compound and a representation of the reference compound; providing a test gene expression profile, comprising a representation of the expression level of a plurality of genes in a test cell exposed to said test compound; comparing said test gene expression profile with said first gene expression profiles; identifying at least one first gene expression profile that is similar to said test gene expression profile; displaying said selected expression profile, and displaying product information related to said selected expression profile.

Another aspect of the invention is a system for performing the method of the invention.

Another aspect of the invention is a computer-readable medium having encoded thereon a set of instructions enabling a computer system to perform the method of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a diagram of an embodiment of a system of the invention.

FIG. 2 depicts a flow diagram illustrating an embodiment of a method of the invention.

FIG. 3 depicts schematic views of the in vivo biology and array processing protocol used in constructing the chemical genomic database of Example 1. (A) Schematic of the in vivo protocol used for large scale analysis of the effects of compounds in rats. (B) Schematic of the large scale array processing procedure. A rectangular box indicates a protocol unit, whereas a diamond-shaped box indicates a quality check of the sample.

FIG. 4 depicts two principle component analysis plots of data from 10,997 microarray experiments from seven different tissues. (A) Analysis of 1,697 control arrays (each represented by a single square); (B) Analysis of 9,300 experimental arrays (various drug-dose-time-tissue combinations, each represented by a single square). PCA was computed based on the log₁₀ signals for the 500 probes with the most variable signal across all 10,997 hybridizations. Therefore each colored square in the graph represents 500 individual measurements of signal intensity. The tissue coloring is as follows, magenta=heart, yellow=brain, red=bone marrow, green=spleen, brown=liver, pink=kidney, and purple=intestine.

FIG. 5 depicts the effects of three anti-cancer drugs on several clinical assays, hematology assays, organ weights, and histopathology observations; (A) Total bilirubin levels (mg/dl) and leukocyte counts (1000/μl) are displayed for carmustine, methotrexate, and thioguanine (y-axis); (B) Log₁₀ ratios for aspartate aminotransferase measured in serum across a total of 891 liver treatments (only 3, 5, and 7 day treatments) for a total of 322 different compounds; (C) Average organ weights for liver and spleen relative to the body weight (average of three animals for each compound); (D) Histopathology findings of liver hepatocyte enlargement.

FIG. 6 depicts plots illustrating the down regulation of the blood cell specific Alas2 in liver correlates with leukocyte count decrease. (A) Log₁₀ signal intensities in whole blood (grey bars) and liver (black bars) are displayed for the 10 RNAs with highest expression in normal blood cells; (B) Chart of Alas2 log₁₀ expression ratio (y-axis) versus leukocyte count log₁₀ ratios (x-axis) across the averages of the liver treatments.

FIG. 7 depicts reticulocyte depletion after anti-cancer drug treatment. (A) Percent reticulocytes in red blood cells from samples treated with the anti-cancer drugs carmustine, methotrexate, and thioguanine at MTD for three days. The average and standard deviation is shown for each drug-dose-time combination and is based on biological quadruplicates; 1000 red blood cells were counted and the percentage of the reticulocytes is reported. (B) Peripheral blood smears stained using the New Methylene Blue dye from treated (carmustine, 16 mg/kg for 3 days) and untreated (vehicle) samples are shown in the left and right images, respectively (representative microscopic fields are shown). Corn oil was used for the vehicle control sample.

FIG. 8 depicts a hierarchical clustering of gene expression level of 73 genes perturbed by these three anti-cancer drugs across a set of 23 liver dose-time conditions. The 73 genes used were selected as being perturbed in a minimum of 8 out of 23 experiments (35% of liver drug-dose-time combinations using carmustine, methotrexate, and thioguanine) and clustered using correlation as the similarity metric (unweighted average method).

FIG. 9 depicts a hierarchical clustering of the top 1000 most variable genes (by standard deviation) across 877 different 3, 5, and 7 day liver treatments. (A) The cluster across all 877 liver treatments versus 1000 genes is shown. (B) Focus on the carmustine subcluster with all the treatments within this cluster (which has an overall correlation coefficient of 0.408).

FIG. 10 depicts an overview of the standard compounds included in database. Details such as number of compounds (cpd), cpd-tissue combinations processed, and structure-activity classification (SAC) are shown for each group.

### DETAILED DESCRIPTION OF THE INVENTION

Definitions:

The term “test compound” refers in general to a compound to which a test cell is exposed, about which one desires to collect data. Typical test compounds will be small organic molecules, typically prospective pharmaceutical lead compounds, but can include proteins, peptides, polynucleotides, heterologous genes (in expression systems), plasmids, polynucleotide analogs, peptide analogs, lipids, carbohydrates, viruses, phage, parasites, and the like.

The term “biological activity” as used herein refers to the ability of a test compound to alter the expression of one or more genes.

The term “test cell” refers to a biological system or a model of a biological system capable of reacting to the presence of a test compound, typically a eukaryotic cell or tissue sample, or a prokaryotic organism.

The term “gene expression profile” refers to a representation of the expression level of a plurality of genes in response to a selected expression condition (for example, incubation in the presence of a standard compound or test compound). Gene expression profiles can be expressed in terms of an absolute quantity of mRNA transcribed for each gene, as a ratio of mRNA transcribed in a test cell as compared with a control cell, and the like. As used herein, a “standard” gene expression profile refers to a profile already present in the primary database (for example, a profile obtained by incubation of a test cell with a standard compound, such as a drug of known activity), while a “test” gene expression profile refers to a profile generated under the conditions being investigated. The term “modulated” refers to an alteration in the expression level (induction or repression) to a measurable or detectable degree, as compared to a pre-established standard (for example, the expression level of a selected tissue or cell type at a selected phase under selected conditions).

The term “correlation information” as used herein refers to information related to a set of results. For example, correlation information for a profile result can comprise a list of similar profiles (profiles in which a plurality of the same genes are modulated to a similar degree, or in which related genes are modulated to a similar degree), a list of compounds that produce similar profiles, a list of the genes modulated in said profile (e.g. a drug signature), a list of the diseases and/or disorders in which a plurality of the same genes are modulated in a similar fashion, and the like. Correlation information for a compound-based inquiry can comprise a list of compounds having similar physical and chemical properties, compounds having similar shapes, compounds having similar biological activities, compounds that produce similar expression array profiles, and the like. Correlation information for a gene- or protein-based inquiry can comprise a list of genes or proteins having sequence similarity (at either nucleotide or amino acid level), genes or proteins having similar known functions or activities, genes or proteins subject to modulation or control by the same compounds, genes or proteins that belong to the same metabolic or signal pathway, genes or proteins belonging to similar metabolic or signal pathways, and the like. In general, correlation information is presented to assist a user in drawing parallels between diverse sets of data, enabling the user to create new hypotheses regarding gene and/or protein function, compound utility, and the like. Product correlation information assists the user with locating products that enable the user to test such hypotheses, and facilitates their purchase by the user.

A “hypothesis” as used herein refers to a testable idea, inspired in by correlation information, regarding an explanation or model of gene or protein function, biochemical or biological function, drug or compound activity or toxicity, absorption, metabolism, distribution, excretion, and the like. Typical hypotheses herein include, without limitation, the identification of a compound or class of compounds as potential lead compounds or drugs, identification of genes or proteins that are characteristic of a disease state or adverse reaction, identification of genes and/or proteins that interact, and the like.

“Similar”, as used herein, refers to a degree of difference between two quantities that is within a preselected threshold. For example, two genes can be considered “similar” if they exhibit sequence identity of more than a given threshold, such as for example 20%. A number of methods and systems for evaluating the degree of similarity of polynucleotide sequences are publicly available, for example BLAST, FASTA, and the like. See also Maslyn et al. and Fujimiya et al., supra, incorporated herein by reference. The similarity of two profiles can be defined in a number of different ways, for example in terms of the number of identical genes affected, the degree to which each gene is affected, and the like. Several different measures of similarity, or methods of scoring similarity, can be made available to the user. For example, one measure of similarity considers each gene that is induced (or repressed) past a threshold level, and increases the score for each gene in which both profiles indicate induction (or repression) of that gene. For example, if gₓ is gene “x”, and P_{Ex} is the expression level of gₓ in an experimental profile, P_{Sx} is the expression level of gₓ in a standard profiles, and p_{T} is a predetermined threshold level, we can define function H for any experimental (“E”) and standard (“S”) profile pair as H_{E,S}=1 when both p_{Ex} and p_{Sx}≧p_{T}, and H_{E,S}=0 when either p_{Ex} or p_{Sx}<p_{T}. Then, a simple similarity score can be defined as N=ΣₓHₓ. This similarity score counts only the genes that are similarly induced in both profiles. A more informative score can be calculated as N′=Σₓ(Hₓ)*|p_{Ex}−p_{Sx}|*(p_{Ex}*p_{Sx})^{-1/2}, which also takes into consideration the difference in expression level between the experimental and standard profiles, for each gene induced above the threshold level. Other statistical methods are also applicable.

The term “product information” as used herein refers to information regarding the availability, characteristics, price, and the like, of a product. Product information can consist of a hyperlink to such information. A product “related to data” refers to a product useful for the further exploration of the gene, protein, system, and/or compound to which the data pertains, or to relationships between the gene, protein, system, and/or compound highlighted in the correlation information. Exemplary products include, for example, bioassay kits and reagents, compounds useful as positive and negative controls, kits for purifying proteins or other biological products, antibodies for determining and/or isolating substances, compounds similar to the test compound useful for further study, additional data regarding gene or protein function and/or relationships (for example, sequence data from other species, information regarding metabolic and/or signal pathways to which the gene or protein belong, and the like), DNA microarrays useful for determining expression of the gene and/or related genes, information and analysis regarding features of a compound that are likely to be responsible for the observed activity, and the like.

The term “hyperlink” as used herein refers to feature of a displayed image or text that provides information additional and/or related to the information already currently displayed when activated, for example by clicking on the hyperlink. An HTML HREF is an example of a hyperlink within the scope of this invention. For example, when a user queries the database of the invention and obtains an output such as a list of the genes most induced or repressed by a selected compound, one or more of the genes listed in the output can be hyperlinked to related information. The related information can be, for example, additional information regarding the gene, a list of compounds that affect gene induction in a similar way, a list of genes having a known related function, a list of bioassays for determining activity of the gene product, product information regarding such related information, and the like.

General Methods:

The system of the invention provides a correlative database that permits one to study relationships between different genes, between genes and a variety of compounds, to investigate structure-function relationships between different compounds, and to facilitate the purchase of products based on such observed relationships. The database contains a plurality of standard gene expression profiles, which comprise the expression level of a plurality of genes under a plurality of specified conditions. The conditions specified can include expression within a particular cell type (for example, fibroblast, lymphocyte, neuron, oocyte, hepatocyte, and the like), expression at a particular point in the cell cycle (e.g., G1), expression in a specified disease state, the presence of environmental factors (for example, temperature, pressure, CO₂ partial pressure, osmotic pressure, shear stress, confluency, adherence, and the like), the presence of pathogenic organisms (for example, viruses, bacterial, fungi, and extra- or intracellular parasites), expression in the presence of heterologous genes, expression in the presence of test compounds, and the like, and combinations thereof. The database can contain expression profiles for a plurality of different species, for example, human, mouse, rat, chimpanzee, yeast such as Saccharomyces cerevisiae, bacteria such as E. coli, and the like. The database preferably comprises expression profiles for at least 10 different genes from a particular organism, more preferably in excess of 500 genes, and can include a substantial fraction of the genes expressed by an organism, such as, for example, about 50%, about 75%, about 90%, or essentially 100%. The standard expression profiles are preferably annotated, for example, with information regarding the conditions under which the profile was obtained. Preferably, the database also contains annotations for one or more genes, more preferably for each gene represented in the database. The annotations can include any available information about the gene, such as, for example, the gene's names and synonyms, the gene's nucleotide sequence the amino acid sequence encoded, any known biological activity or function, any genes of similar sequence, any metabolic or protein interaction pathways to which it is known to belong, a listing of assays capable of determining the activity of its protein product, and the like.

The database contains interpretive gene expression profiles and bioassay profiles for a plurality of different compounds that comprise a representation of a compound's mode of action and/or toxicity (“drug signatures”), and can include experimental compounds and/or “standard” compounds. Drug signatures provide a unique picture of a compound's comprehensive activity in vivo, including both its effect on gene transcription and its interaction with proteins. Standard compounds are preferably well-characterized, and preferably exhibit a known biological effect on host cells and/or organisms. Standard compounds can advantageously be selected from the class of available drug compounds, natural toxins and venoms, known poisons, vitamins and nutrients, metabolic byproducts, and the like. The standard compounds can be selected to provide, as a set, a wide range of different gene expression profiles. The records for the standard compounds are preferably annotated with information available regarding the compounds, such as, for example, the compound name, structure and chemical formula, molecular weight, aqueous solubility, pH, lipophilicity, known biological activity, source, proteins and/or genes it is known to interact with, assays for detecting and/or confirming activity of the compound or related compounds, and the like. Alternatively, one can employ a database constructed from random compounds, combinatorial libraries, and the like.

The database further contains bioassay data derived from experiments in which one or more compounds represented in the database are examined for activity against one or more proteins represented in the database. Bioassay data can be obtained from open literature and directly by experiment.

Further, the database preferably contains product data related to the compounds, genes, proteins, expression profiles, and/or bioassay data otherwise present in the database. The product data can be information regarding physical products, such as bioassay kits and reagents, compounds useful as positive and negative controls, compounds similar to the test compound useful for further study, DNA microarrays and the like, or can comprise information-based products, such as additional data regarding gene or protein function and/or relationships (for example, sequence data from other species, information regarding metabolic and/or signal pathways to which the gene or protein belong, and the like), algorithmic analysis of the compounds to determine critical features and likely cross-reactivity, and the like. The product information can take the form of data or information physically present in the database, hyperlinks to external information sources (such as a vendor's catalog, for example, supplied via the Internet or CD-ROM), and the like.

The database thus preferably contains five main types of data: gene information, compound information, bioassay information, product information, and profile information. Gene information comprises information specific to each included gene, and can include, for example, the identity and sequence of the gene, one or more unique identifiers linked to public and/or commercial databases, its location on a standard array plate, a list of genes having similar sequences, any known disease associations, any known compounds that modulate the encoded protein activity, conditions that modulate expression of the gene or modulate the protein activity, and the like. Product information comprises information specific to the available products, and varies depending on the exact nature of the product, and can include information such as price, manufacturer, contents, warranty information, availability, delivery time, distributor, and the like. Bioassay information comprises information specific to particular compounds (where available), and can include, for example, results from high-throughput screening assays, cellular assays, animal and/or human studies, biochemical assays (including binding assays and enzymatic assays) and the like. Compound information comprises information specific to each included compound, such as, for example, the chemical name(s) and structure of the compound, its molecular weight, solubility and other physical properties, proteins that it is known to interact with, the profiles in which it appears, the genes that are affected by its presence, and available assays for its activity. Profile information includes, for example, the conditions under which it was generated (including, for example, the cell type(s) used, the species used, temperature and culture conditions, compounds present, time elapsed, and the like), the genes modulated with reference to a standard, a list of similar profiles, and the like. The information is obtained by assimilation of and/or reference to currently-available databases, and by collecting experimental data. It should be noted that the gene database, although large, contains a finite number of records, limited by the number of genes in the organisms under study. The compound database is potentially unlimited, as new compounds are made and tested constantly. The profile database, however, is still larger, as it represents information regarding the interaction of a very large number of genes with a potentially infinite number of different compounds, under a variety of conditions:

Experimental data is preferably collected using a high-throughput assay format, capable of examining, for example, the effects of a plurality of compounds (preferably a large number of standard compounds, for example 10,000) when administered individually or as a mixture to a plurality of different cell types. Assay data collected using a uniform format are more readily comparable, and provide a more accurate indication of the differences between, for example, the activity of similar compounds, or the differences in sensitivity of similar genes.

The system provides several different ways to access the information contained within the database. An operator can enter a test gene expression profile into the system, cause the system to compare the test profile with stored standard gene expression profiles in the database, and obtain an output comprising one or more standard expression profiles that are similar to the test profile. The standard expression profiles are preferably accompanied by annotations, for example providing information to the operator as to the similarity of the test profile to standard profiles obtained from disease states and/or standard compounds. The test gene expression profile preferably includes an indication of the conditions under which the profile is obtained, for example a representation of a test compound used, and/or the culture conditions.

The output preferably further comprises a list of the genes that are modulated (up-regulated or down-regulated) in the test gene expression profile, as compared with a pre-established expression value, a pre-selected standard expression profile, a second test gene expression profile, or another pre-set threshold value.

The output is preferably hyperlinked, so that the operator can easily switch from, for example, a listing of the similar standard expression profiles to a listing of the modulated genes in a selected standard expression profile, or from a gene listed in the test profile to a list of the standard expression profiles in which the gene is similarly modulated, or to a list of the standard compounds (and/or conditions) which appear to modulate the selected gene. The output can comprise correlation information that highlights features in common between different genes, targets, profiles, compounds, assays, and the like, to assist the user in drawing useful correlations. For example, the output can contain a list of genes that were modulated in the user's experiment with a selected compound: if a plurality of the genes are indicated as associated with liver toxicity, the system can prompt the user that the compound is associated with a toxic drug signature, and prompt the user to continue with the next compound. Conversely, the output could indicate previously unnoticed associations between different pathways, leading the user to explore a hitherto unknown connection. The output preferably includes hyperlinks to product information, encouraging the user to purchase or order one or more products from a selected vendor, where the product(s) relate specifically to the focus of the database inquiry and the correlation information that results, and is presented back to the user to facilitate hypothesis generation. For example, the output can provide links to products useful for confirming the apparent activity of a compound, for measuring biological activity directly, for assaying the compound for possible side effects, and the like, prompting the user to select products useful in the next stage of experimentation.

The system is preferably provided with an algorithm for assessing similarity of compounds. Suitable methods for comparing compounds and determining their morphological similarity include “3D-MI”, as set forth in copending application U.S. Ser. No. 09/475,413, incorporated herein by reference in full, Tanimoto similarity (Daylight Software), and the like. Preferably, the system can be queried for any compounds that are similar to the test compound in structure and/or morphology. The output from this query preferably includes the corresponding standard expression profiles (or hyperlinks to the corresponding standard expression profiles), and preferably further includes a listing, description, or hyperlink to an assay capable of determining the biological activity of the standard and/or test compound.

Thus, for example, if the user inputs an experimental expression profile resulting from incubation of test cells with a particular experimental compound, the user can obtain an output comprising an estimate of the quality of the data, an identification of the genes affected by the compound, a listing of similar profiles and the conditions under which they were obtained (for example, the compounds used), and a list of compounds having a structural similarity. The output can be provided in a hyperlinked format that permits the user to then investigate and explore the data. For example, the user can examine which genes are modulated, and determine whether or not the genes have yet been characterized as to function or activity, and under what conditions each gene is modulated in a similar fashion. Alternatively, the user can compare the profile obtained with the profile of a desired outcome, for example comparing the profile obtained by incubation of diseased or infected tissue with a test compound against a profile obtained from healthy (unperturbed) tissue. Alternatively, the user can compare the profile with the profiles obtained using standard compounds, for example using a drug of known activity, mechanism of action, and specificity, thus determining whether the test compound operates by a different mechanism, or if by the same mechanism whether it is more or less active than the standard. Additionally, the user can compare the structure of the test compound with the structures of other compounds with similar profiles (to determine which structural features of the compounds are common, and thus likely to be important for activity), or can compare the compound's profile with the profiles obtained from structurally similar compounds in general.

The system can be configured as a single, integrated whole, or can be distributed over a variety of locations. For example, the system can be provided as a central database/server with remotely-located access units. The remote access units can be provided with sufficient system capability to accept and interpret test gene expression profiles, and to compare the test profiles with standard gene expression profiles. Remote units can further be provided with a copy of some or all of the database information. Optionally, the remote system can be used to upload test gene expression profiles to the central system to update the central database, or a “private” database supplementary to the main database can be stored in or near the remote unit.

Further, the system can be divided into “vendor” and “client” portions, separating segments of the system into any economically useful subsets, in which interaction between a vendor unit and a client unit is monitored and/or governed by the client's state. For example, the system can be configured to treat a primary database as a vendor unit, and remote access units as client units. The vendor database can be configured to respond to a plurality of different permission levels, wherein lower permission levels are granted access to only a restricted subset of the available data, with successively higher levels obtaining access to greater amounts of data. For example, the lowest permission level can provide access only to publicly-available gene sequences and public annotations, without correlations to compounds or profiles. The client system in such cases can be equipped to provide statistical analysis of the profile generated by the user, the ability to identify genes within the profile, and the ability to compare gene sequences for similarity. In this case, the interaction between client unit and vendor unit can be limited to access to the publicly-available gene sequences, which can be provided electronically, or exchanged via a storage medium (for example, using CD-ROM, DVD, or the like). The bulk of the vendor database (for this permission level) can be pre-installed at the client location, avoiding the need to download large amounts of data (for example, limiting downloads only to updates). This level can be essentially unrestricted, i.e., allowing public access without need for a pre-existing vendor-client relationship.

An intermediate permission level can provide access to a larger subset of data, for example including links to some or all of the available profile and compound data in addition to the information provided to the lower permission level. In this case, the interaction between client and vendor systems occurs contemporaneously or after a client account is established, determining the level of access to be granted the client. If conducted electronically, the interaction is preferably accomplished through means of a secure transaction, to ensure that neither the vendor data nor the client queries are rendered non-confidential. Such transactions can be conducted, for example, by adapting the systems and methods disclosed in U.S. Pat. No. 5,724,424, incorporated herein by reference in full. The data in this case can be limited to compounds that are publicly known (for example, commercially available, or disclosed in patents or the like) and profile data related to those compounds. Alternatively, the system can be arranged so that the client obtains access only to a specific field, for example, profiles related to diabetic conditions, autoimmune conditions, cancer, and the like. For cases of intermediate permission, the vendor system can filter output before it is transmitted to the client system, to insure that only the permitted degree of information is distributed. The vendor system can also filter input, to insure that vendor system resources are not consumed in preparing answers that cannot be delivered to the client system.

At the penultimate permission level, the client is granted access to all data in the database except for data that is proprietary, restricted, or exclusively granted to another client. The ultimate permission level may be available only to the vendor itself, or can be made available to one or more clients if no exclusivity is granted to clients.

Additionally, the system can include provisions for accepting new data from a remote client, for example, to enable a user to store his or her own data on the vendor server. Access to such client data can be restricted to only the same client, or can be made available to all clients or a subset thereof (for example, in exchange for a credit or other privilege).

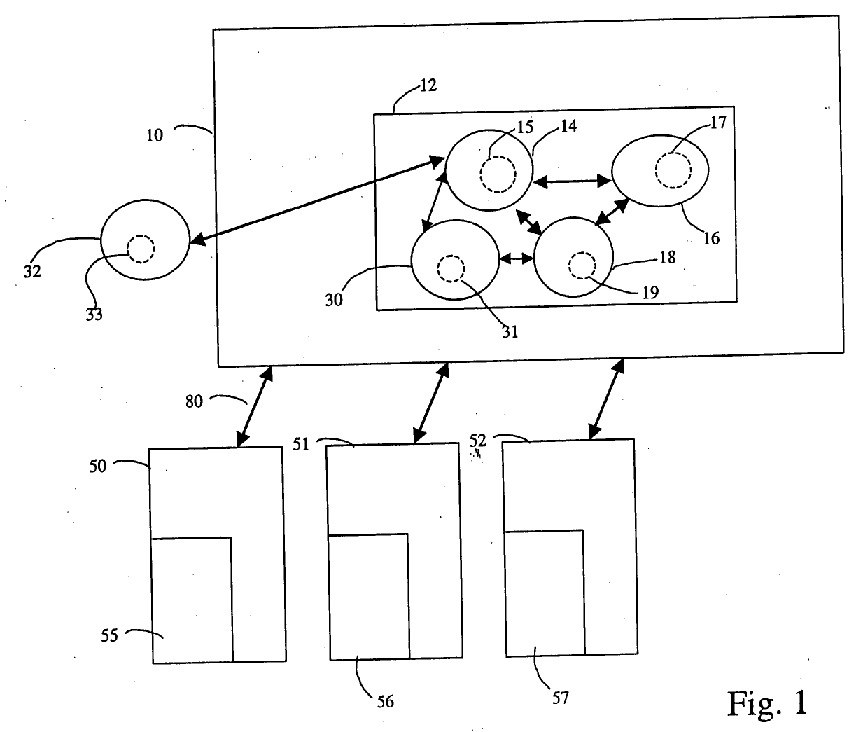

FIG. 1 illustrates a system of the invention, comprising vendor server 10 containing vendor database 12. Vendor database 12 in turn contains a genomic database 14, a compound database 16, and a profile database 18, which in turn contain optional private (user) databases 15, 17, and 19. Alternatively, the private databases can be physically located outside the vendor databases, for example, elsewhere within the vendor system or maintained in parallel within the user's site. The vendor databases can further comprise a product database 30 maintained within the vendor system, and/or an external product database 32 linked to the vendor system. The product databases can contain information regarding products available from the vendor, a third-party vendor, or both. One or both of the product databases can further comprise user-specific data (31, 33) such as, for example, user account information (account number, format preferences, shipping addresses, prior order history, authorization level, and the like), the user's notes or annotations regarding particular products, and the like. The product databases are preferably provided with hyperlinks that facilitate user purchases of the products displayed. The vendor system is connected to a plurality of user systems 50, 51, 52, which in turn contain individual user databases 55, 56, 57. The user systems can communicate with the vendor system by any convenient medium, including, without limitation, direct connection, distributed network (LAN or WAN), internet connection, virtual private network (VPN), direct dial-in, and the like. The hardware employed for use in the method of the invention can comprise general-purpose computers, for example currently-available personal computers and workstations, or special-purpose terminals designed for this application.

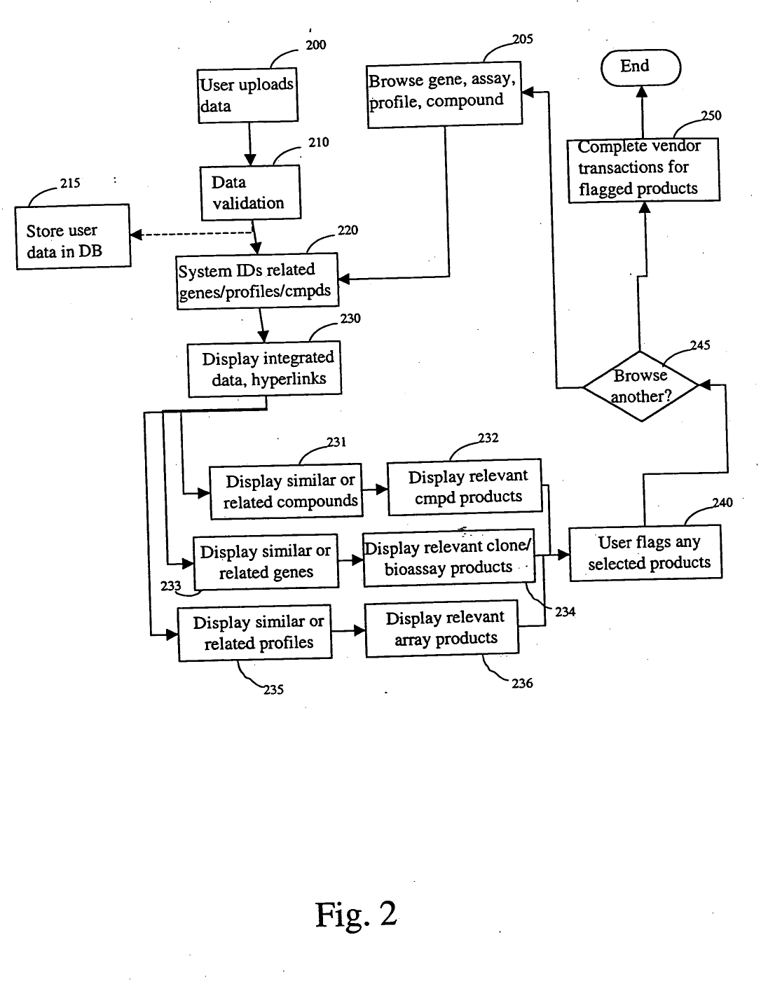

FIG. 2 illustrates a simple flow diagram for an embodiment of the invention. The user may begin by uploading data into the system 200 (or otherwise acquiring profile data), or alternatively may simply begin by browsing 205 for a gene, compound, or profile of interest already present in the system. If new data is added, the data can optionally be evaluated and validated 210. Optionally, the new data can be uploaded to the primary database, as either a public or private addition, or can be stored in the user portion of the system 215. After data validation (if any), the data is examined by the system, and the genes and profile identified 220. This result is displayed 230, along with hyperlinks to related product information. Preferably, the results are displayed in a manner that highlights correlations between similar expression profiles, the profiles of similar compounds, the profiles of related genes, and the like. The user can then select more information regarding one or more related compounds 231, genes 233, profiles 235, and the like, at which point the system can display relevant compound products 232, relevant clones and/or bioassay products 234, or relevant array products 236. The output display preferably facilitates selection of relevant products by the user, flagging selected products 240 (for example, adding them to a “shopping cart” system). The user can then select 245 a path of inquiry, and search for compounds of similar structure, morphology, or activity (in terms of profile), for selected genes or genes of similar sequence or known function, or for similar profiles 205. These results are displayed 230, and the user invited to continue browsing until finished. Alternatively, the user can pre-select various forms of output, for example, selecting to have the initial data display include a listing of similar compounds linked to displays of their profiles, or a listing of the experimental profile along with a list of similar profiles ranked by degree of similarity. Alternatively, the user can upload a chemical structure (whether real or hypothetical), and obtain a display of a predicted profile extrapolated from the profiles of morphologically similar compounds.

These methods can be conducted on a single computer, or can be distributed over a plurality of computers. For example, steps 200, 205 and 230 can occur on a remote computer (at the user site), while other steps occur on a local computer or computers, or at another remote site distinct from the user's site (the vendor server).

Data concerning experimental pharmaceutical compounds and their biological activity are extremely sensitive, valuable and confidential. In embodiments that include computers or other hardware at a plurality of locations, it is presently preferred to include some provision for security, for example by regulating access or by means of encrypted commands and results. Suitable methods are known in the art, including, for example, public key encryption and SSL (secure socket layer) connections. Alternatively, rather than reporting gene expression data in terms of absolute expression, one can report the data in terms of differences from a given standard. Thus, if gene “A” has an arbitrary standard expression value of 56 (in arbitrary units), and in an experimental profile gene “A” is expressed at a level of 97, the data for gene “A” can be reported as expression of 41 rather than 97. A different standard level can be established for each gene employed, essentially forming an encoding profile. A plurality of different encoding profiles can be established and enumerated for each user and shared by secure means, with the user and vendor simply indicating which profile (by number) is used for each transmission. Further, one can express the data in terms of other arithmetic functions and combinations of functions of an encoding profile, as long as the original data can be unambiguously retrieved by the authorized party. For example, the encoding transform for a particular encoding profile can specify that data for the first gene is expressed as the difference between the experimental and profile values, while data for the next gene is expressed as a percentage of the profile value, while data for the third gene is expressed as the difference between the third experimental value and the second experimental value, and the like. If additional security is desired, one can establish encoding profiles and transforms that change depending on other parameters, for example by date, by user number, by time of file modification, by number of data sets, and the like, and combinations thereof. Alternatively, one can specify a large number of available encoding profiles, and specify in advance a random sequence of profiles to employ, avoiding the identification of any profile during transmission of data.

The general method of the invention as described above is exemplified below. The following examples are offered by way of illustration and not by way of limitation. The disclosure of all citations in the specification is expressly incorporated herein by reference.

### EXAMPLES

### Example 1

### Construction of a Chemical Genomic Database

This example describes the construction of a chemogenomic database based on DNA microarray analysis of gene expression profiles of selected tissues from compound treated rats.

A. Overview

The effectiveness of a chemogenomic database increases with thoughtful standard compound selection and data reproducibility, which, in turn depends largely on standardized protocols. As described in detail below there were several protocols whose full standardization resulted in the generation of consistent and high quality expression profile data from DNA microarrays. These include the standard compound and dose selection protocols, the in vivo biology (e.g. exposure time and animal data collection protocols) and the microarray processing protocols (e.g. RNA isolation, cRNA preparation, array hybridization, and data-uploading).

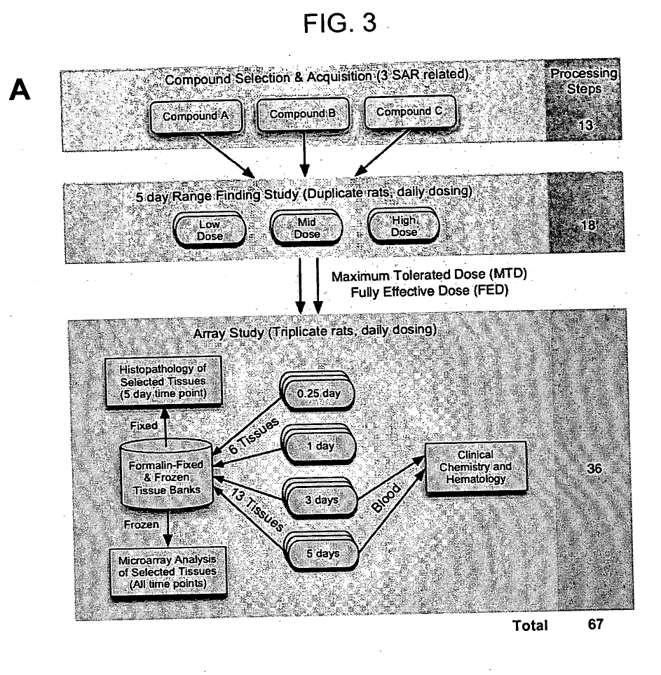

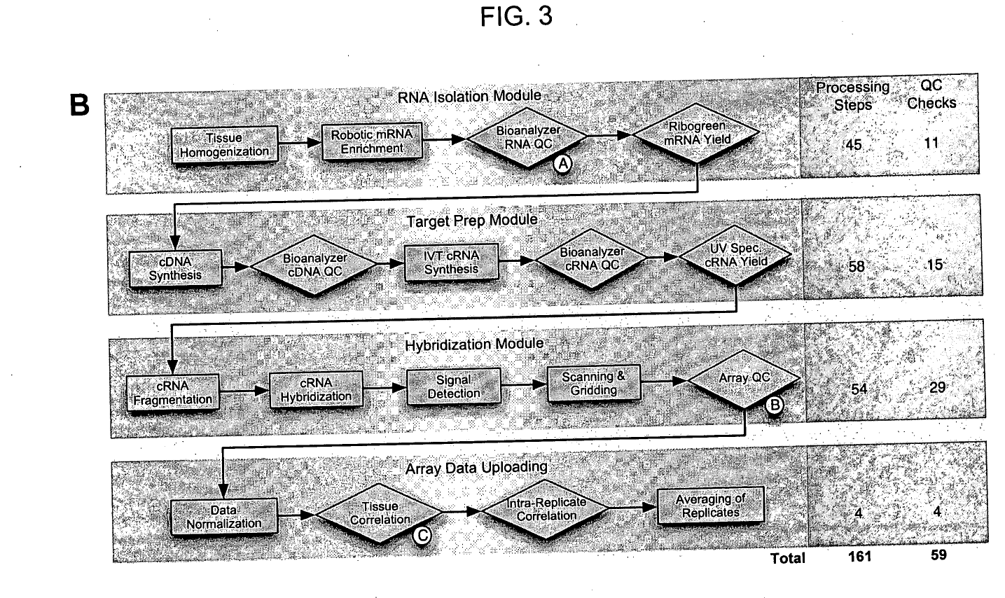

FIG. 3 depicts a schematic view of the in vivo biology and array processing protocol used in constructing the chemical genomic database of Example 1. FIG. 3A shows the three in vivo protocol modules, with the number of processing steps listed for each protocol. The three protocols used were: (1) Compound selection and acquisition; (2) 5 day Range Finding Study; and (3) the Array Study. At least three SAR-related compounds (depicted in FIG. 3A as compounds A, B, and C) were selected whenever three such related compounds are available, each member of a set is processed on different study days to eliminate any study day bias. Compounds were tested during the Range Finding Study at three different doses, the low, mid, or high dose (with daily repeat dosing). The identified Maximum Tolerated Dose (MTD) and the estimated Fully Effective Dose (FED) was then used for the Array Study at four different time points, 0.25, 1, 3, and 5 days (with daily repeat dosing for the latter two). A maximum of 13 tissues were collected per drug-dose-time condition and stored in a central frozen tissue bank and a formalin-fixed tissue bank. Six tissues were harvested from the two earlier treatment conditions (the 0.25 and 1 day), these included liver, kidney, heart, bone marrow, and a sixth tissue chosen based on literature studies indicating an organ of toxicological concern or a pharmacological target organ. Of the two later treatment conditions, a panel of several clinical chemistry and hematology parameters was measured (see, Table 2). Histopathology analysis was performed on the tissue of interest (one or more of 13 tissues) generally using only the 5 day treatment condition using a standard vocabulary and severity scale (Table 4). FIG. 3B depicts a schematic of the large scale array processing procedure which is divided into four different protocol modules with the number of processing and quality control steps listed for each protocol (two columns on the right). The different protocols are further divided into sub-protocols, as represented by the different boxes. A rectangular box indicates a protocol unit, whereas a diamond-shaped box indicates a quality check of the sample.

The database system was implemented as a 3-tier platform: (1) a database; (2) a web server; and (3) a client application. The database used was a standard relational database that references both simple data types and binary objects. The web server was the middle tier and acted the container for the application. The client application was implemented as a web browser that rendered server generated XML into dynamic HTML thereby creating a rich client experience.

The network used may be of any type (e.g. LAN, WAN, etc.) that supports standard internet communication protocols. Generally, the hardware requirements are flexible and depend on the size of the database. Preferably, a high-end server for the relational database is used to achieve optimal performance of the database.

B. In Vivo Biology Protocols

This section describes the in vivo biology protocols, including the standard compound dosing of the rats and the tissue harvesting protocols as outlined in FIG. 3A.

1. Compound Selection

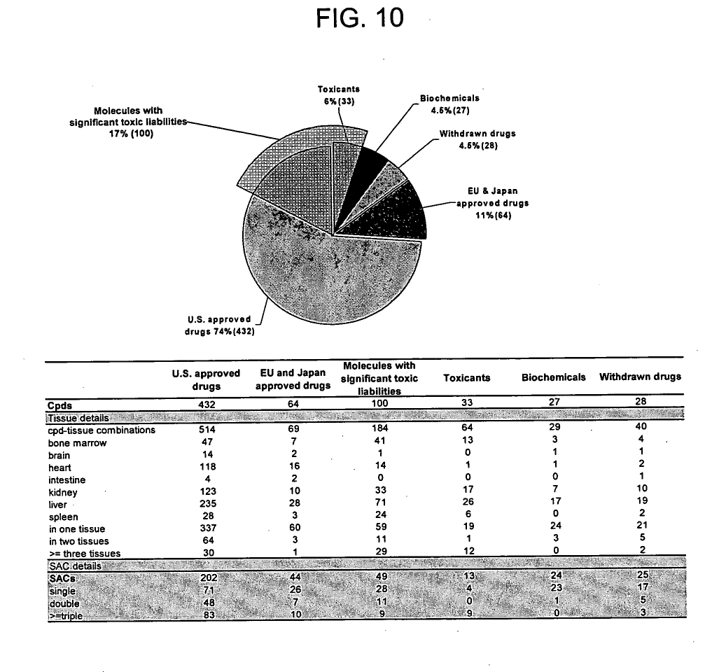

A list was assembled containing all approved U.S., European and Japanese pharmaceuticals, all compounds withdrawn by regulatory authorities, and biochemical agents that are not intended to be human pharmacological agents but have defined molecular targets in the biochemical and toxicological literature. Also included were known toxicants, drawn from well characterized literature examples, resulting in a final list of standard compounds including about 2000 approved and withdrawn drugs, biochemical reagents, and toxicants. As a principle criterion for selection of compounds for inclusion within the database, a group of at least three similar compounds, related by structure, pharmacologic activity, toxicity, and/or mechanism, was selected whenever possible. By selecting groups of related compounds, a fuller representation of their pharmacological effects are more easily identified because the resultant gene expression profiles (i.e. transcription patterns) may be more easily correlated with the true effect of the compound class rather than an event unique to a single compound. A detailed overview of the standard compounds with information such as the distributions of structure activity subclasses and tissues is shown in FIG. 10.

The standard compounds used for the studies described here were obtained from a variety of sources including the three major sources, Sequoia Research Products, Sigma-Aldrich, and Fluka, which provided 85% of the compounds. The synthesis of a small number of compounds was commissioned from outside laboratories. With the exception of a few compounds of microbial fermentation origin, the purity of each compound was >90% based on the certificate of analysis provided by the compound suppliers. Of the 584 compounds studied, the median purity was 99.4% and average purity was 98.7%. Purity confirmations were conducted on each compound sample by independent LC/MS analysis coupled with in-line evaporative-light-scattering detection. Compound samples of less than 95% purity, by LC/MS, were further confirmed by NMR spectroscopy.

2. Animal Details

Male Sprague-Dawley (Crl:CD®(SD)|GS BR) rats (aged 6-8 weeks and weighing 200-260 g), were purchased from Charles River Laboratories (Wilmington, Mass.). They were housed in plastic cages for 1 week for acclimation to the laboratory environment of a ventilated room (temperature, 22° C.±3° C.; humidity, 30-70%; light/dark cycle, 12 h/d, 6:00 am-6:00 pm) until use. Certified Rodent Diet #5002 (PMI Feeds Inc.) and chlorinated tap water was available ad libitum. The 0.25 and 1 day time points were harvested starting at 1:00PM and completed within 1-2 hours, whereas the 3 and 5 day time points were harvested starting at 7:00AM and completed within 2-4 hours; all harvests used an appropriately staggered schedule so that the harvest times are accurate to +/−30 min. of the designed dose-to-harvest interval.

3. Dose Selection—Range-Finding (RF) Study

When comparing the effects of diverse compounds, it is preferable to administer them at doses that are as biologically and toxicologically equivalent as possible. At least two doses were selected for each standard compound rat dosing experiment. The higher dose, which is targeted to be the maximum tolerated dose (MTD), is intended to elicit an equivalent general toxicological response, e.g., consistent reduced weight gain relative to the control group. This dose is anticipated to induce mild gross toxicity but also to identify target organ toxicity for a wide variety of compounds that vary in terms of intrinsic efficacy, pharmacodynamics, and pharmacokinetics. The lower dose, the fully effective dose (FED), is chosen to elicit the pharmacologic effects of a given drug, which contributes to the understanding of the mechanism of action (MOA) of a compound of interest.

Setting dose for RF study: A thorough search of several literature sources was performed to identify information related to each standard compound, including: acute toxicity, LD₅₀, route of administration for clinical compounds, or typical exposure routes for toxicants. For dose setting purpose and analyzing literature data, species were preferred in the following order: rat is preferred over mouse which is chosen over any other species. A disease model was chosen over a pharmacokinetic study, if possible. Studies in which animals were chronically dosed are more favored than those in which a single dose is administered. Finally, a study which uses a disease model that mimics the human indication for the compound was given more consideration than an alternative disease model. The two most important parameters for dose setting are body weight change and clinical observations. A typical control rat will gain between 16-20% of body weight in the six days of the study.

The Range-Finding (RF) studies (see also FIG. 3A) were designed to estimate the upper limits of non-lethal toxicity (i.e. the MTD) by identifying a test compound dose that would produce an approximate 50% decrease in the rate of growth relative to control animals after five days of repeat dosing (with sacrifice on the 6^{th} day). For the RF study three dose levels were administered daily for five consecutive days via the route of administration (ROA) that corresponds to that by which humans receive the drug. Compounds were typically administered orally (PO) (83%), intravenously (IV) (9.4%), subcutaneously (SC) (5.7%), or intraperitoneally (IP) (2%). If the compound was a toxicant or a biochemical standard, it was administered orally. For oral dosing, the vehicle choice largely depends on the solubility of the compound in water. Water-soluble compounds are administered in water. Insoluble compounds were administered in either corn oil or 0.5% carboxymethylcellulose (CMC) using the best literature recommendation as a guide. IV administered compounds were usually dissolved in saline and SC administered compounds in corn oil. The highest dose that the rats receive was the Maximum Tolerated Dose (MTD) which is defined from the initial RF study and described in detail below.

Lower Dose (FED): The lower dose (FED) was defined as the dose that induces maximal pharmacologic effects in an animal model of the disease for which the drug or compound is most frequently used therapeutically. The FED was derived from the literature and is, whenever possible, identical to the dose used to successfully treat a relevant rat model of disease. In many cases, the essential criterion of a precise disease model, ROA, duration of dosing, and species could not all be met in a single study. For these situations, a systematic and hierarchical selection procedure was developed for proper dose selection from the literature. There were three considerations for ranking the literature: species, dosing regimen, and disease model. In the complete absence of relevant literature information, or when the compound was a toxicant or a biochemical standard, the FED was defined as 10% of the high dose (MTD).

At the FED, it was assumed that most compounds exert their pharmacological effects with minimal toxicological consequences. However, it should be noted that many drugs will have no discernible therapeutic effects in a wild-type, disease-free rat. For example, there is no molecular target for antibiotics in such rats. Conversely, some compounds with narrow margins of safety (e.g. chemotherapy agents) will likely induce some level of toxicity at pharmacologic doses, impairing the ability to cleanly separate mechanism of toxicity from mechanism of pharmacology.

High Dose (MTD) range finding study: MTD was defined as the dose that allows a male Sprague-Dawley rat to achieve a 5-10% increase in its body weight over the course of 5 consecutive once daily dosings. Control vehicle-treated animals typically gained between 16-20% of their weight over the same time period, thus the maximum tolerated dose reduced the rate of growth of the treated animals by about 50%, but did not cause severe clinical signs of toxicity.

To determine the MTD, rats were dosed for five consecutive days at three dose levels (two animals per range finding group) based on the LD₅₀ of the compound for the relevant ROA using the RF study. The three dose levels were the LD₅₀ dose (high dose), the LD₅₀/2 (mid dose), and the LD₅₀/4 (low dose). To ensure that the same criterion were used for setting the high array dose for each compound studied, a system was devised for interpreting the results from this low animal number in a standardized way. Clinical observations of toxicity and body weight gain were used in an algorithm to derive the high dose, the MTD. Briefly the algorithm evaluated the following: If the body weight gains were>10% for the highest dose (during the same time period, vehicle-treated animals increase their weight by 16-20%) and the dose used was the LD₅₀ derived from the best-available literature, that dose was defined as the MTD. Otherwise, a dose-response in body weight change with respect to dose administered must be observed AND at least one dose must produce a 5-10% average body-weight gain. For cases when LD₅₀ information was not available from the literature, doses were set by using the LD₅₀ for other ROAs or another species allometrically scaled to rat according to the following formula (see, Wallace-Hayes, A. Principles and Methods of Toxicology (2001)):

$${Dose}_{rat} = [ {{Dose}_{species} ( {Weight}_{species )} ]}^{1 / 4} / {Weight}_{rat} .$$

If this information could not be found, the RF dose was based on a combination of toxic dose low values and curated toxicity information from the literature.

Where initial RF studies could not produce a clear determination of MTD, and compound supplies and solubility were not limiting factors then the range finding study was repeated at newly selected doses based on the findings of the initial range finding study. In some cases, where compound safety is very high, solubility and compound supply may limit the ability to deliver an MTD. In these cases a maximum feasible dose (MFD) was selected and was typically set to 2000 mg/kg. Estrogenic compounds were found to frequently have these features.

4. Array Study—Tissue Harvesting

Array studies using each standard compound were performed once enough information was obtained from the RF studies to accurately set the high doses. Tissue samples for microarray gene expression analysis were harvested from test compound-treated and vehicle control-treated rats after 0.25 day, 1 day, 3 days and 5 days of exposure with daily dosing. In a few studies (1.8%) 7 days of exposure was substituted for 5 days of exposure. The time points were chosen to capture the immediate effects of a compound (0.25 day), effects that occur within the first day after a single dose (1 day), and to understand how the compound-induced events change over repeated administration (3 and 5 days) and to allow a projection of effects that might be expected to occur with long term exposure.

In addition to the microarray analysis, the standard compound treated tissue samples were also used to carry out bioassays including: clinical chemistry, hematology, organ weight, and gross and histological pathology (see FIG. 3A).

In the same manner as for the RF experiments, standard animal laboratory guidelines were adhered to and the rats were the same strain (Sprague-Dawley), sex (male) and age (6 to 8 weeks old). Additionally, environmental conditions including food, water, bedding quality, day-night cycle, temperature, and humidity were tightly controlled as summarized below. To eliminate extraneous sources of variation in gene expression and microarray data, all animal dosing and necropsy occurred in a 2-4 hr window relative to the day-night cycle, depending on study size. The dosing of animals was staggered based on intended harvest order to ensure that sacrifice occurred within 30 minutes of the recorded time point. To ensure proper management of harvested tissue, each tube was barcoded, and each technician harvested tissues from one animal at a time verifying that the animal tag number matched the number on the tubes before starting. Sample tubes were labeled with barcodes prior to sacrificing animals to allow faster harvest, and thereby ensuring shorter lag times (<30 minutes) between death and tissue freezing in order to prevent RNA degradation. The tissue harvest order was such that the more perishable organs, as determined in preliminary studies, were harvested first (those tissues are usually allowed less than 5 minutes between sacrifice and snap freezing; spleen is the most sensitive tissue). 6 mm disposable biopsy punches (#REF 33-36 Miltex, Inc. Bethpage, N.Y.) were used to obtain tissue samples of approximately 100 mg. The tissue samples were placed in 4 ml internally threaded cryogenic vials (#430490 Corning, Inc. Corning, N.Y.) for sample storage. These cryogenic vials are used because they allow sample storage of 100 samples in a standard 3 inch freezer inventory box and are large enough to allow homogenization of the tissue sample within the same tube after addition of the lysis buffer. After snap freezing in liquid nitrogen, each barcoded tube was scanned to record its position in a barcoded storage box. This position list is used for sample tracking. Blood was harvested at the time of sacrifice, for the 3, or 5 day animal necropsy. For each compound treatment the following 13 tissues are usually collected: liver, kidney, heart, brain, intestine, fore stomach, blood, spleen, bone marrow, lung, muscle, lung, and reproductive organ,

To allow better statistical assessment of the data, each “Array Study” dose-time experiment was executed in biological triplicate (See e.g., Cutler, D. J., M. E. Zwick, M. M. Carrasquillo, C. T. Yohn, K. P. Tobin, C. Kashuk, D. J. Mathews, N. A. Shah, E. E. Eichler, J. A. Warrington, and A. Chakravarti. 2001. High-throughput variation detection and genotyping using microarrays. Genome Res 11: 1913-1925; and Ramakrishnan, R., D. Dorris, A. Lublinsky, A. Nguyen, M. Domanus, A. Prokhorova, L. Gieser, E. Touma, R. Lockner, M. Tata, X. Zhu, M. Patterson, R. Shippy, T. J. Sendera, and A. Mazumder. 2002. An assessment of Motorola CodeLink microarray performance for gene expression profiling applications. Nucleic Acids Res 30: e30), whereas each RNA sample representing a particular animal was hybridized only once. This choice was based on analysis of biological and technical replicates that indicated relatively little incremental value is gained by running more than three experiments per dose-time combination, whereas the overall animal, compound, and microarray costs increase substantially.

5. Results

The in vivo rat dosing protocol for each standard compound contained two separate studies, a Range Finding (RF) study and an Array Study. For the RF study, three dose levels of each compound were chosen after careful review of the literature and are administered once daily for five consecutive days in duplicate (total of 6 animals). The estimated dose selection approach led to successfully identifying the desired dose 62% of the time (423 out of 681 RF studies conducted), and 25% of the RF studies had to be repeated before defining the MTD. In 12% of the RF experiments, a Maximum Feasible Dose (MFD) was determined rather than an MTD. In certain cases, an MFD, a dose greater than FED but less than MTD was used when the constraints of compound supply, cost, and solubility limit the use of higher doses. For example, most compounds with an MTD>2000 mg/kg were dosed at an MFD. Twenty five percent of all the compounds were administered at the MFD for their high dose during the array study protocol.

The use of the dosing regimen based on the RF study allowed the Array Study that succeeded in inducing the desired 5-10% body weight increase upon compound administration in 74% of the compound studies. Of the remainder, 8% were dosed at the MFD, whereas the remaining 18% of compounds failed to suppress the weight gain. Even though 18% of drugs failed to suppress weight gain sufficiently to be considered to be at their MTD (estrogenic compounds are a notable example), the data was still incorporated into the database because body weight criterion is not the only indicator of toxicity. Data from bioassays including clinical pathology, necropsy information, organ weight change, and histopathology were also evaluated when analyzing the toxicity of a particular compound.

C. Microarray Processing Protocols

A highly standardized microarray processing protocol was established containing a total of 88 quality control checkpoints to control the fate of samples from compound treated rats being moved along the entire processing pipeline from compound treatment to processed microarrays. This tightly controlled process assured that only samples of good quality were promoted to the next step, and therefore only excellent quality expression profiling data entered the correlative database.

1. Microarrays

The Uniset Rat I Expression (RU1) and Uniset Human I Expression BioArrays used for the experiments described here were purchased from Amersham Biosciences (Piscataway, N.J.). The RU1 BioArray contained 30-mer probes for 9,911 (8,565 probes used for data analysis) unique sequences representing 9,641 unique genes. The human BioArray, used in a few investigative studies, contained 30-35-mer probes for 9,995 unique sequences representing 9,921 unique genes.

2. Automated Isolation and Purification of RNA using the MagNA Pure Robot

Poly A(+)-RNA from both cell culture and tissue samples was isolated using the MagNA Pure LC robot (Roche, Basel, Switzerland) in combination with the MagNA Pure LC mRNA Isolation kit I and II (Roche, Basel, Switzerland) for cells and tissues, respectively. It was found that in comparison to manually isolated RNA samples, that the automated isolated procedure described here resulted in much greater accuracy and reproducibility at a lower cost per sample.

Cell culture lysates were retrieved from the −80° C. freezer and allowed to thaw at room temperature. Once thawed the samples were drawn 5-6 times through a 20-gauge needle using a 3-ml syringe to break up cell debris. Omission of this syringing step would result in highly variable yields. 300 μl of each sample was loaded into one of the wells of the MagNA Pure (capacity of the robot: 32 samples using a 32-well plate), which is programmed to extract RNA using oligo-dT selection technology into a final elution volume of 100 μl.

Tissue samples were completely homogenized directly from ˜100 mg punches stored on dry ice prior to application of lysis buffer to a final concentration of 65 mg tissue per ml of buffer. After complete homogenization, using disposable Omni Tip Disposable Generator Probes (Omni Inc, Warrenton, Va.) and before loading of the samples into the 32-well MagNA Pure plate, the samples were drawn up 5-6 times through a 20-gauge needle attached to a 3-ml syringe to ensure that tissue pieces or clumps were removed from the lysate for robotic processing. Tissue sample processing was performed in duplicate wells (loading 150 μl of homogenized sample to each well) of the MagNA Pure LC, which is programmed to extract mRNA using the oligo-dT selection method into a final elution volume of 100 μl.

Poly A(+) RNA sample concentration was performed manually using a standard ethanol precipitation protocol in the presence of glycogen (50 μg/ml). After precipitation the final purified RNA sample was resuspended in 7 μl DEPC-H₂O and quantified using a Ribogreen high-range assay (Molecular Probes) on the Wallac Victor2 Fluorometer (Perkin-Elmer, Fremont, Calif.). Additionally, the integrity of each RNA sample was determined, by comparison to historical standards (no gross degradation should be visible as suggested by clear 18S and 28S peaks on top of a hump of complex RNA, with lower amounts of RNA below 18S than under and above the 18S peak), using the Agilent 2100 BioAnalyzer (Agilent Technologies, Palo Alto, Calif.) in combination with the RNA 6000 Nano Lab Chip kit (Agilent Technologies).

To study the impact of RNA quality obtained using two different RNA isolation procedures on the downstream processing and reproducibility of array quality, the MagNA Pure LC RNA isolation system was compared to a standard manual RNA isolation procedure. It was found that the coefficient of variation of the automated sample set was approximately one-half that observed for the manually isolated RNA. A similar improvement was observed when studying the percentage of false positives observed in self-self analysis; 5% of the elements displayed values that differed by more than 2-fold using a manual RNA preparation, whereas only 0.5% showed this difference using the automated RNA procedure. Generally, the manually prepared sample set is noisier and yields a higher percentage of false positives when compared to the automated sample set.

Furthermore, an analysis of the actual RNA product using an electropherogram produced by a capillary electrophoresis system (Agilent 2100 BioAnalyzer, Palo Alto, Calif.), showed that the RNAs purified using either procedure still contained a substantial portion of ribosomal RNA. The ribosomal RNA content of the automated RNA sample is 33-53% (43±10%, sample N=192), whereas for the manual sample it is 15-47% (31±16%, sample N=18). However, the enriched RNA isolated using the automated procedure is more consistent (CV=23.3%) from sample to sample (as measured by rRNA contamination) when compared to manually prepared samples (CV=51.6%) and consequently is of superior quality for microarray experiments. Lastly, this automated RNA isolation procedure results in several fold increase in throughput at a much lower cost per sample.

3. Automated cRNA Preparation

The methods used for cRNA preparation (cDNA synthesis, cRNA preparation, and cRNA purification) are essentially as described in the CodeLink™ manual v2.1 as supplied by Amersham Biosciences (Piscataway, N.J.) using the Qiagen BioRobot 9604 (Valencia, Calif.). cDNA synthesis, cRNA preparation, and cRNA purification were completely processed in a 96-well format using the automated Qiagen BioRobot 9604 procedure. 0.6-20 μg of enriched RNA from different tissue sources were added to a reaction mixture in a final volume of 12 μl, containing bacterial control RNA (1.5 pg FixA, 5 pgYjeK, 5 pg AraB, 15 pg EntF, 50 pg FixB, 150 pg HisB, 500 pg LeuB, 1500 pg gnd) and 1.0 μl of 100 pmol/μl T7-(dT)₂₄ oligonucleotide primer (Proligo, Boulder, Colo.). The T7-(dT)₂₄ oligonucleotide primer used, is an HPLC purified 63-mer with the sequence: 5′-GGCCAGTGAATTGTAATACGACTCACTATAGGGAGGCGGTTTTTTTTTTTTTTTTTT TTTTTT-3′. (SEQ ID NO: 1) The mixture was incubated for 10 min at 70° C. and chilled on ice. On ice, 4 μl of 5x first-strand buffer, 2 μl 0.1 M DTT, 1 μl of 10 mM dNTP mix and 1 μl Superscript™II RNaseH-reverse transcriptase (200 U/μl) were added to the mixture to make a final volume of 20 μl. The mixture was incubated for 1 hr at 37° C. Second-strand cDNA was synthesized in a volume of 150 μl, containing 92 μl nuclease-free water, 30 μl of 5x second-strand buffer, 3 μl of 10 mM dNTP mix, 4 μl of Escherichia coli DNA polymerase I (10 U/μl) and 1 μl of RNase H (2 U/μl) for 2 hr at 16° C. The cDNA was purified using a Qiagen QIAquick purification kit, and completely dried down using a Speed-Vac concentrator (45° C.) for 2 hr. The dried product was resuspended in IVT reaction mix containing 3.0 μl of nuclease-free water, 4.0 μl 10x reaction buffer, 4.0 μl 75 mM ATP, 4.0 μl 75 mM GTP, 3.0 μl 75 mM CTP, 3.0 μl 75 mM UTP, 7.5 μl 10 mM Biotin 11-CTP, 7.5 μl 10 mM Biotin 11-UTP and 4.0 μl enzyme mix. The reaction mix was incubated for 14 hr at 37° C. using an MJ Research 96-well PTC-200 Thermal Cycler (MJ Research, Waltham, Mass.), before the cRNA was purified using a Qiagen RNeasy® kit. The resulting cRNA yield was quantified using the 96-well KC4 UV spectrophotometer (BIO-TEK Instruments Inc., Winooski, Vt.) at a wavelength of 260 nm. Conformance of the cRNA sample to historical size distributions (the bulk of the cRNA product should be between 400 and 800 bases in size) was confirmed using the Agilent 2100 BioAnalyzer (Agilent Technologies, Palo Alto, Calif.). Samples not near the historical norm were reprocessed starting from tissue or RNA.

4. Hybridization

12.5 μg of cRNA sample was fragmented in 40 mM Tris-acetate (TrisOAc) pH7.9, 100 mM KOAc and 31.5 mM MgOAc at 94° C. for 20 min. This typically resulted in a fragmented cRNA with a size range between 100 to 200 bases. 10 μg of the fragmented cRNA was used for hybridization of each Rat-Unset I (RU1) Expression BioArray (Amersham Biosciences, Piscataway, N.J.) in a volume of 260 μl, containing 78 μl of CodeLink™ Hyb buffer component A and 130 μl of CodeLink™ Hyb buffer component B (Amersham Biosciences, Piscataway, N.J.). The hybridization solution was denatured at 90° C. for 5 min then chilled on ice. The sample was vortexed at maximum speed for 5 sec and centrifuged at maximum speed for 5 min before 250 μl of the solution was injected into the inlet port of the flex-hybridization chamber, and placed in a CodeLink™ 12-slide shaker tray. The hybridization ports were sealed with 1 cm sealing strips (Amersham Biosciences, Piscataway, N.J.), and the shaker tray(s) containing the slides was loaded into a New Brunswick Innova™ 4080 shaking incubator, with the hybridization chambers facing up. Slides were incubated for 20 hr at 37° C., while shaking at 300 rpm.

5. Post-Hybridization Signal Detection

The 12-slide shaker tray was removed from the shaker, and the hybridization chamber removed from each slide. Each slide was placed into the BioArray Rack of the Parallel Processing Tool (Amersham Biosciences, Piscataway, N.J.) and incubated with 0.75×TNT (0.075 M Tris-HCl, pH7.6, 0.1125 M NaCl, 0.0375% Tween-20) at 46° C. for 1 hr. The BioArray rack was moved from the TNT containing reservoir to the small reagent reservoir containing 1:500 dilution of streptavidin-Alexa 647 (Molecular Probes, Eugene, Oreg.). The signal was developed for 30 min at room temperature, before the reaction was stopped and slides were washed four times for 5 min each in TNT buffer (0.1 M Tris-HCl, pH7.6, 0.15 M NaCl, 0.05% Tween-20) using a large reagent reservoir. The slides were rinsed in ddH₂O with 0.05% Tween-20 twice for 5 sec each before they were dried by centrifugation with a Qiagen Sigma 4-15C centrifuge (Valencia, Calif.) using a swinging bucket rotor (2×96) for exactly 3 min at 2000 rpm (acceleration at position 9 and deceleration at position 9). The dried slides were stored in light protective slide boxes at room temperature prior to scanning. These last steps of the process were found to be critical to achieving high quality data. Each time and temperature should be adhered to exactly, with absolutely no deviation in time and no more than 1° C. deviation in temperature. Consequently, it was necessary to process no more than 20 slides (2×10 slides) at one time.

6. CodeLink™ BioArray Scanning and Analysis

The Axon GenePix Scanner (Axon Instrument, Union City, Calif.) was calibrated using the “Calibration Slide” supplied by Axon Instrument with GenePix 4.0 at 635 nm using the “Calibration System”. After calibration of the scanner, all processed slides were scanned with the laser set to 635 nm, the photomultiplier tube (PMT) voltage to 600 and the scan resolution to 10 microns. For consistency all slides were scanned the same day of color development, within an hour after dry spinning them and the data was analyzed using the CodeLink™ Expression Analysis Software version 2.2.25, (Amersham Biosciences, Piscataway, N.J.).

7. Array Normalization

Prior to statistical computation, the spot reading data was normalized. For this purpose a nonlinear normalization procedure similar to the centralization approach reported described previously (Zien, A., T. Aigner, R. Zimmer, and T. Lengauer. 2001. Centralization: A new method for the normalization of gene expression data. Bioinformatics) was used. The normalization procedure uses an algorithm that assumes that in general, for an array with many probes, the majority of the signal represents genes that have unchanged expressions compared to controls, with the extreme values representing the true biology of the process and not some artifact due to measurement noise. The algorithm does not make the assumption that the true mRNA abundance being measured is linearly proportional to the spot reading signal or any assumption about the error distribution of such signals or their differential. Rather, the assumption of unchanged signals representing the bulk of the signal measurements is used to center a nonlinear curve fit to a reference template. This reference template is constructed for a large set of time matched, same tissue and same vehicle, control arrays, computing a median log signal for each probe. The replicate size of this set is adequate to ensure very small random error in the ensemble signal level for each reference probe. The curve fit corrects for some array processing problems, such as partial signal saturation, and improves the overall quality of the data compared to simple linear normalization methods. Essentially the curve tracks the mode of the signal distribution for sets of genes against the expected value for that set.

8. Mean Log Ratio and Significance Calculations

Notation: First define indices: g=1, . . . ,G genes, k=1, . . . K (drug/dose/time) treatment conditions, and i=1, N_{X} replicate measurements with X_{gki}=log (Signal) for each gene in each condition (assumes Signal values were already array-wise normalized).

Data were taken with respect to an overall measurement context defined by microarray type and model, animal strain, tissue, treatment time, and administration mode (vehicle/route). For each measurement context a set of log (signal) values for vehicle-treated control measurements was obtained, C_{gi}, j=1, . . . N_{c}. Such control measurements reflect the reference gene-expression levels that form the basis for comparison of each treatment condition.

Statistically, it was assumed that the observed measurements have a Gaussian (i.e. normal) distribution, X_{gki}˜N(μ_{Xgki}, σ²_{Xgk}), and the control measurements have a Gaussian distribution, C_{gki}˜N(μ_{Cgki}, σ²_{Cgk}). Without loss of generality, and for clarity, the subscript g is suppressed in the following description. Log base 10 is used everywhere for consistency.

Log Ratios: To compare samples, expression levels in the compound treated group were matched to a control group and the relative expression values were computed. For statistical leverage this differential expression was then converted to a log ratio. The estimate of mean log ratio (for any particular gene) for condition k was calculated by formula,
Dₖ=\overline{X}ₖ−\overline{C}.

Significance of results: an estimate of the standard deviation of a population was calculated using the standard deviation formula for an estimate around an estimate of its true value, and denoted as the standard error of the estimate. For simple replication the standard error of a mean of n replicate measurements with individual standard deviation a was calculated by σ/\sqrt{n}. The CodeLink™ single color array platform produces resulted in two populations, the treated and control groups. The standard error of D_{gk} around its estimated true value was calculated using the formula:

$$SE ( {D}_{k} ) = \sqrt{\frac{{σ}_{Xk}^{2}}{{N}_{X}} + \frac{{σ}_{C}^{2}}{{N}_{C}}}$$

Substitute an estimate for the values of the σs. Assuming that σ²⁼σ²_{Xgk}=σ²_{Cg}, the statistical technique of pooling estimates of variance was used to combine the individual variance estimates for each group using the formula:

$$SE ( {D}_{k} ) = \sqrt{\frac{( {N}_{X} - 1 ) {S}_{Xk}^{2} + ( {N}_{C} - 1 ) {S}_{C}^{2}}{{N}_{X} + {N}_{C} - 2}}$$

The degrees of freedom for the denominator of the classic Student's two-sample t was calculated using df=N_{X}+N_{C}−2.

However, if the assumption of equal variances for controls and experimental animals was questionable, then a safer version of the t-test, the Welch t-test was used which estimates the variances separately for each group to calculate a t-test denominator by formula

$$SE ( {D}_{k} ) = \sqrt{\frac{{S}_{Xk}^{2}}{{N}_{X}} + \frac{{S}_{C}^{2}}{{N}_{C}}}$$

with estimated (and possibly non-integer) degrees of freedom

$$df = \frac{{( {S}_{{X}_{g}}^{2} / {N}_{X} + {S}_{C}^{2} / {N}_{C} )}^{2}}{\frac{{( {S}_{{X}_{g}}^{2} / {N}_{X} )}^{2}}{{N}_{X} - 1} + \frac{{( {S}_{C}^{2} / {N}_{C} )}^{2}}{{N}_{C} - 1}}$$

In either case, the t-statistic computed as

$$T = \frac{{D}_{k}}{SE ( {D}_{k} )}$$

was obtained from a standard t-table based on df degrees of freedom to tabulate the corresponding p-values, confidence intervals, etc.

Estimates of SE based only on the data for each situation are very specific and may not have enough information to provide adequate estimates of error. In the most common case where N_{X}=3, there are only 2 degrees of freedom for S²_{X}, which leads to imprecise estimates, and in particular, sometimes the estimated sigma can be too small, leading to false positives. Additionally, if only one observation is available, there is no unbiased estimate of sigma. A global estimate of σ could be used, assuming that for each gene, the σs are constant across conditions. This may not be reasonable as different treatments may affect the biological variability of the gene, so it would be better to have a method that allows for this possibility. To address these issues, an Empirical Bayes approach was used similar to that described in Baldi and Long, “A Bayesian framework for the analysis of microarray expression data: regularized t-test and statistical inferences of gene changes,” Bioinformatics 17: 509-519 (2001), but modeled gene by gene (since we have many replicate sets). It was assumed that true sigmas for each situation are drawn according to the appropriate conjugate prior (an inverse chi-square distribution), and fit the scale and shape coefficients of that distribution to the available data. The improved and stabilized EB estimates of the standard deviation for each situation were of the form

$${S}_{X}^{′ 2} = \frac{{ν}_{X} {σ}_{X0}^{2} + ( {N}_{X} - 1 ) {S}_{X}^{2}}{{ν}_{X} + {N}_{X} - 1} ,$$

where σ²_{XO} is the pooled global variance estimate, and v_{X} is the degrees of freedom for the contribution of the global estimate. Note that v_{X} does not grow to infinity even if the global set of data gets large, it reflects amount of variability in specific situations. But the extra degrees of freedom were sufficient to give the local variance estimates much more stability. The hyper parameters for each situation were estimated based on the data for many replicate situations for each tissue separately. The control variability has its own set of hyper parameters that were used to calculate improved control variance estimates by a parallel formula

$${S}_{C}^{′ 2} = \frac{{ν}_{C} {σ}_{C0}^{2} + ( {N}_{C} - 1 ) {S}_{C}^{2}}{{ν}_{C} + {N}_{C} - 1}$$

The hyper parameters for controls may be similar to those for the experimental situations, but may not be identical, since the control sets are not triples of experiments run at the same time, but collected as sets over a wider time period, etc. Based on these improved estimates, the standard error was calculated as:

$$SE ( {D}_{k} ) = \sqrt{\frac{{S}_{Xk}^{′ 2}}{{N}_{X}} + \frac{{S}_{C}^{′ 2}}{{N}_{C}}}$$

with degrees of freedom

$$df = \frac{{( {S}_{{X}_{g}}^{′ 2} / {N}_{X} + {S}_{C}^{′ 2} / {N}_{C} )}^{2}}{\frac{{( {S}_{{X}_{g}}^{′ 2} / {N}_{X} )}^{2}}{{ν}_{X} + {N}_{X} - 1} + \frac{{( {S}_{C}^{′ 2} / {N}_{C} )}^{2}}{{ν}_{C} + {N}_{C} - 1}}$$

For each expression and gene, CodeLink™ displayed the log ratio, standard error and p value for the expression changes between the treated and control arrays.

9. Array Quality Control Assessment Procedure

The array quality control assessment procedure consisted of five stringent rounds of array quality assessments, and was used to determine whether the data quality was sufficient for an array to enter the final database.

(1) Round one focuses on the overall array quality and is based on un-normalized array data, values such as: mean signal, background, and log dynamic range values. This round quickly identifies bad arrays, and typically passes greater than 97% of input arrays.

(2) Round two does not result in failed arrays, but rather bins them based on whether they require additional reviewing due to the fact that some values are on the borderline. Round two is also based on un-normalized array data and on average results in placing about 10% of arrays into the review bin.

(3) Round three, based on normalized data, requires a visual inspection of all arrays within the review bin and is based on normalized data. During this round each array is inspected for its pattern relative to a reference set. The inspection uses two different tools, the false color image and the scatter plot. If the color pattern of the false color image is uneven, or the scatter plot noisy and or deviates substantially from the 45° line, an array is considered of poor quality and does not enter the database; usually a new array is prepared from cRNA remaining from the cRNA synthesis step. This visual round of quality control assessment results in an overall success rate of greater than 93%; it improves the data substantially as poor quality data is not allowed to enter the database.

(4) During round four of the quality control assessment, poorly processed arrays are identified and failed using correlation criterion. The correlation (across all probes) of an array of interest to the reference control tissue array is computed. If the correlation between the test array and the reference control tissue array is below 0.8 AND the correlation of the test array to any other (other tissue types) reference control tissue array is above 0.8, an array is considered a failed array. In addition, if the correlation between the test array and the reference control tissue array is below 0.65, an array is considered of poor quality and is failed immediately. Exceptions to these two rules are bone marrow and spleen samples. Bone marrow and spleen tissues are highly correlated to each other and the criterion described above cannot be applied at this stage of the process.

(5) Round five finally assesses the overall quality of the biological triplicate by calculating the correlation between each of the arrays within the set to each other across all probes. Any poorly correlated array (with a CC<0.8) within a replicate is easily identified and excluded from the set.

As shown in FIG. 3, the standardized in vivo and array processing protocols entails 228 different processing steps. Furthermore, 88 quality control metrics determine whether a sample proceeds from one step to the next. Key quality control metrics are highlighted by diamonds in FIG. 3B. Seven quality control metrics that were determined to contribute significantly to the quality of the database are listed in Table 1.

TABLE 1

| | | | # QC |
|---|---|---|---|
| | QC Metric | Key metrics / pass if | Steps |
| Compound | Compound purity and identity | LC/MS anatysis of identify | 4 |
| acquisition | | Average purity > 90%, | |
| In vivo | Prelimiary MTD dose determnation | Achieve MTD = 5-10% body weight gain in the | 2 |
| RF Study | | absence of clinical signs | |
| In vivo | Strictly followed tissue harvest protocot adherence | Dosing Schedule: 6:30 am + 2-4 hr | 21 |
| Array study | to all time and temperature requirements | Tissue apecitic harvest schedule: Blood +≦ 2 | |
| | | minutes | |
| | | Sacrifice schedule: <30′ between sacrifice and last | |
| | | sample in LN2: barcoded semples | |
| mRNA | mRNA yield/concentration | >0.095 μg/μl (6 μl) | 11 |
| isolation | Ribosomal contemination | <45% | |
| | Cap electrophoresis profile | Not degraded: vs. historical standard | |
| cRNA target | cRNA yield | ≧0.53 μg/μl and > μg | 15 |
| preparation | Cap etectrophoresis / cDNA size | >500 bp | |
| Array hybridiza- | Strict Protocol adherence to all time and temperature | cRNA Fragmentation: EXACTLY 94C° for 20′ | 29 |
| tion and color | requirements | Color development: EXACTLY 30′ | |
| development | Assurance of sample integrity | Process maximum of 20 arrays / batch | |
| Array DC | Ave Norm Background | <2.0 | |
| round 1 (raw | Med. Signal to Threshold | >0.8 | |
| signal data) | Fraction of Signal below Threshold | <0.6 | |
| | Log Dynamic Range | >1.0 | |
| round 3 | Scatter plot to reference standard | Clean and straight 45° line | |
| (normalized data) | False Color maps | Evenly colored | |
| round 4 | Correlation analysis to tissue reference standard | Correlation to reference control standard >= 0.8 | 4 |
| (normalized data) | | (across all probes) | |
| round 5 (biolo- | Correlation analysis of each array within a replicate | Correlation within a replicate >= 0.8 (across all | |
| gical triplicate) | | probes) | |

For every RNA sample, the quantity and quality were analyzed and used as criteria to determine whether a sample is adequate for further processing. An RNA sample moved on if the integrity was confirmed, the ribosomal content was below 50%, and the concentration was greater than 0.095 μg/μl in a total volume of 6 μl. For the cRNA preparation, the concentration had to be greater than 0.529 μg/μl and the yield at least 13 μg for a sample to be hybridized. At the array level, the array quality was assessed using a procedure consisting of several metrics for stringent quality control assessments including e.g. the correlation coefficient (of log₁₀ normalized signal across all probes) for each array versus a tissue standard formed by averaging 20-100 control tissue samples and the pair-wise correlations (of log₁₀ normalized signal across all probes) for each array within its dose-time-tissue-drug matched replicate (usually three samples) were computed. These correlations needed to be greater than 0.8 for array data to be included in the database.

10. Results: Process Performance and Improvement

To better understand the modifications implemented in the hybridization module and its performance over time, a variance analysis study was performed. Since pooling the samples at the post cRNA preparation stage reveals information about variance introduced by the tool and hybridization process, cRNA (RNA isolation and cRNA preparation) was prepared from control livers and pooled before hybridization onto six individual arrays using the standard procedure. This experiment was done twice, spaced apart by 17 months. To summarize, the array and hybridization variance dramatically improved over a period of 17 months from 42.4% to 19.8%. This improvement was attributed to the various process improvements described above, such as various protocol improvements including changes to the cRNA fragmentation, i.e. at 94° C. (±1° C.) for exactly 20 minutes, and strict adherence to time and temperature during the optimized color development steps.

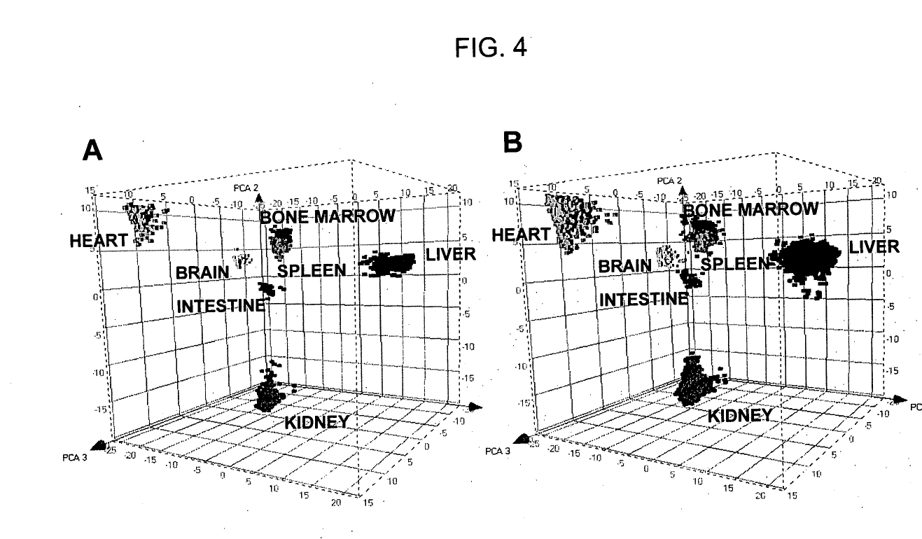

To visualize the quality of the accumulated gene expression data in the context of the entire database, principal component analysis (PCA) was employed using the top 500 most variable probes (probes with the highest standard deviation in log₁₀ signal intensity) across a total of 10,997 control and experimental arrays derived from seven different tissues and 3200 drug-dose-time combinations. As shown in FIG. 4A, the control arrays cluster tightly within their individual and separated “tissue clouds”, with few out-of-cloud arrays. Extending this analysis using the experimental arrays from drug treated animals (FIG. 4B) results in somewhat more dispersion in the clouds of arrays and is consistent with the expected impact of drugs on gene expression. These results support the conclusion that the above-described protocols result in a high quality and consistent chemical genomic database that may be used to carry out correlative analysis of compound treated expression profiles.

### Example 2

### Correlating Compound Effects on Gene Expression with Traditional Clinical Chemistry Bioassays including Hematology Panel, Relative Organ Weights, and a Fixed Histopathology

Clinical bioassay outcomes for each drug were also compiled in the chemogenomic database of Example 1. This feature allows subsequent data mining efforts where traditional toxicology bioassays such as increases in bilirubin may be associated with the gene expression profile changes in the same animals. In one preferred method of correlative analysis, the expression data may be queried with a classification hypothesis (e.g. “Compounds resulting in bile duct hyperplasia versus those that do not.”). Optimized classification algorithms (e.g. Support Vector Machines) may be used to derive short drug signatures that allow prediction of traditional clinical markers and histopathologies based solely on gene expression profiling data. This drug signature approach reduces the complexity of thousands of gene expression changes down to a handful of predictive biomarkers for a number of biologically meaningful endpoints.

Blood based bioassays have been the most common measurements used to determine outcomes both during drug development and as part of clinical practice. In order to connect the new technologies of gene expression measurements to the well understood measurements used in traditional drug and chemical toxicological testing, values for these traditional bioassays were collected for the compound treated tissues harvested in constructing the chemogenomic database as described in Example 1. The effect of 584 compounds on these parameters is summarized in Table 2.

A large proportion of the compounds (328 of 584) caused significant alterations in at least one of the 19 clinical chemistry measurements. Changes in the serum levels of ALT were quite common, with 88 compounds causing a significant increase (outside the 95% tolerance interval) in this blood marker of liver injury. 122 of 584 compounds caused significant increases in at least one of the 14 hematology parameters, while 219 of them caused significant decreases in at least one of the parameters.

TABLE 2

| | Controls | | | | |
|---|---|---|---|---|---|
| | 95% Toler. | | | Compounds | |
| Assay (units) | Avg. | Lower | Upper | Incr. | Decr. |
| Clinical Chemistry | | | | | |
| BLOOD UREA NITROGEN (mg/dl) | 15.3 | 9.76 | 23.3 | 59 | 46 |
| CREATININE (mg/dl) | 0.20 | 0.08 | 0.46 | 94 | 0 |
| GLUCOSE (mg/dl) | 160 | 108 | 230 | 16 | 15 |
| ASPARTATE AMINOTRANSFERASE (u/l) | 87.8 | 54.0 | 138 | 82 | 26 |
| ALANINE AMINOTRANSFERASE (u/l) | 54.4 | 30.2 | 93.5 | 88 | 48 |
| ALKALINE PHOSPHATASE (u/l) | 370 | 206 | 636 | 25 | 36 |
| TOTAL BILIRUBIN (mg/dl) | 0.19 | 0.07 | 0.43 | 56 | 14 |
| SODIUM (meq/l) | 143 | 128 | 160 | 2 | 1 |
| POTASSIUM (meq/l) | 6.2 | 4.41 | 8.55 | 11 | 9 |
| CHLORIDE (meq/l) | 100 | 90.6 | 111 | 9 | 8 |
| PHOSPHORUS (mg/dl) | 11.4 | 8.45 | 15.3 | 12 | 30 |
| TOTAL PROTEIN (g/dl) | 5.95 | 5.08 | 6.96 | 25 | 35 |
| ALBUMIN (g/dl) | 4.14 | 3.49 | 4.89 | 15 | 74 |
| CHOLESTEROL (mg/dl) | 70.4 | 45.3 | 107 | 48 | 55 |
| CREATINE PHOSPHOKINASE (u/l) | 400 | 64.7 | 1570 | 21 | 0 |
| LACTATE DEHYDROGENASE (u/l) | 245 | 25.9 | 1190 | 9 | 0 |
| CARBON DIOXIDE (mmol/l) | 29.3 | 19.6 | 42.8 | 0 | 32 |
| URIC ACID (mg/dl) | 1.33 | 0.30 | 4.68 | 3 | 6 |
| LIPASE (u/l) | 10.1 | 5.95 | 16.3 | 42 | 1 |
| Number of compounds with no significant changes: | | | | 256 | |
| Hematology | | | | | |
| LEUKOCYTE COUNT (×10³/ul) | 12.6 | 4.4 | 31.5 | 5 | 29 |
| ERYTHROCYTE COUNT (×10⁶/ul) | 5.5 | 4.6 | 6.7 | 60 | 29 |
| HEMOGLOBIN (g/dl) | 13.5 | 11.5 | 15.7 | 56 | 34 |
| HEMATOCRIT (%) | 34.8 | 29.2 | 41.3 | 54 | 32 |
| MEAN CORPUSCULAR VOLUME (fl) | 63.0 | 57.6 | 68.8 | 4 | 7 |
| MEAN CORP. HEMOGLOBIN (pg) | 24.4 | 21.4 | 27.8 | 3 | 18 |
| MEAN CORP. HEMOGLOBIN CONC. (g/dl) | 38.8 | 35.3 | 42.5 | 7 | 19 |
| PLATELET COUNT (×10³/ul) | 1142 | 467 | 2597 | 0 | 11 |
| NEUTROPHIL (%) | 8.8 | 2.2 | 28.3 | 39 | 10 |
| LYMPHOCYTE (%) | 89.7 | 78.3 | 102 | 0 | 92 |
| ABSOLUTE SEG. NEUTROPHIL (/ul) | 1117 | 185 | 4775 | 33 | 17 |
| ABSOLUTE LYMPHOCYTE (/ul) | 11245 | 3989 | 28029 | 0 | 37 |
| ABSOLUTE MONOCYTE (/ul) | 234 | 36.3 | 1024 | 9 | 107 |
| ABSOLUTE EOSINOPHIL (/ul) | 218 | 38.2 | 876 | 0 | 145 |

| | |
|---|---|
| Number of compounds with no significant changes: | 406 |

Since many of the compounds were dosed at their MTD, a biologically and statistically significant effect on clinical pathology parameters was frequently observed, and a wide diversity of effects was observed among the compounds. It appears from examining many safe and effective drugs, that about 44% have no effect on any clinical chemistry endpoint and 70% have no effect on any hematological endpoint. Liver damage (as indicated by a rise in ALT levels) was a fairly common finding in rats treated with high doses of compounds, as 88 of 584 (15%) of the compounds that were tested were associated with increases of serum ALT. Kidney damage, as indicated by increases in blood urea nitrogen (BUN) or creatinine (CRE) occurred for about 59 (0.10%) or 94 (16%), respectively, of 584 of compounds evaluated. Effects on white blood cell parameters were also common, for example, 92 of 584 compounds (16%) decreased the percentage of circulating lymphocytes, and 29 (5%) of 584 of compounds decreased the number of leukocytes.

In addition, the collection of this type of traditional bioassay data in a uniform way for such a large numb er of diverse compounds is in itself a valuable reference for establishing the level of concern regarding apparent toxicities in a drug candidate. For example, during the development of a new drug targeted towards an existing marketed class, it can be accurately benchmarked against existing drugs in the database that have already been profiled.

The data in Table 2 illustrate a diverse representation of chemical-induced toxicities as produced using the protocols described herein. Furthermore, toxicities to several organs are evident with some compounds; whereas other compounds produced little or no toxicity based on classical markers. The lack of apparent toxicity in a number of compounds is important since many of the methods of data mining applied to this dataset rely on classifying normal from injured gene expression patterns.

The ability to correlate traditional clinical bioassay data with gene expression data is one of the key useful features of the integrated correlative database of the present invention. For example, the ability of compounds to increase or decrease the weight of an organ relative to body weight was evaluated since these measurements are also used as an indicator of organ-specific damage in preclinical chemical and drug testing. As shown in Table 3, the liver was the most frequent target of compound induced organ weight changes, as 71 of 578 compounds (12.3%) were associated with increased relative liver weights.

TABLE 3

| | Controls | | | | | | |
|---|---|---|---|---|---|---|---|
| | | | 95% Toler. | | | | |
| | Avg. Rel. | Std | Lower | Upper | Compounds | | |
| Tissue | Weight (%) | Dev. | Limit | Limit | Incr. | Decr. | N |
| FORESTOMACH | 0.161 | 0.081 | 0.377 | −0.055 | 8 | 0 | 568 |
| GLANDULAR STOMACH | 0.378 | 0.084 | 0.604 | 0.152 | 9 | 3 | 568 |
| GONADS | 0.968 | 0.471 | 2.233 | −0.298 | 0 | 0 | 568 |
| HEART | 0.390 | 0.052 | 0.530 | 0.249 | 23 | 3 | 578 |
| INTESTINE | 0.304 | 0.143 | 0.689 | −0.081 | 4 | 0 | 568 |
| KIDNEYS | 0.923 | 0.086 | 1.153 | 0.692 | 44 | 4 | 578 |
| LIVER | 4.753 | 0.464 | 6.001 | 3.505 | 71 | 10 | 578 |
| LUNGS | 0.642 | 0.147 | 1.037 | 0.246 | 6 | 0 | 568 |
| SPLEEN | 0.251 | 0.049 | 0.382 | 0.121 | 37 | 18 | 570 |

| | | |
|---|---|---|
| Number of compounds with no significant changes: | 396 | 578 |

The first two data columns show the average and standard deviation of organ weights expressed as a percentage of terminal body weight for the same 837 control animals (3, 5, or 7 days of daily dosing). The last three columns indicate the number of compounds that increase or decrease the relative organ weight beyond the bounds of the 95% tolerance intervals of the controls, and N, the number of compounds where data was available for each organ; N is not identical for each organ because in a few isolated cases data was not collected at the time of sacrifice. The averages and standard deviations for each organ were calculated assuming a normal distribution. For experimental treatments at 3, 5, or 7 days, the relative organ weight data from triplicate animals representing particular drug-dose-time combinations were averaged. For purposes of comparison, if the average for a particular drug at either of the final two time points fell outside the 95% tolerance limits of the controls (at least 2.688 standard deviations away from the mean of the controls), then that drug was deemed positive for an organ weight change. For reference, the mean body weight of these control animals was 253±23 grams across all control animals.

Formalin fixed tissue sections were examined at the 5-day time point (see FIG. 3A). A standardized fixed organ-specific histopathology vocabulary was established by a board certified pathologist and used to score formalin-fixed hematoxylin-eosin stained tissue sections from control and vehicle treated rats. The vocabulary is indicated in the table, along with the corresponding incidences observed in control and compound treatments. Only observations with positive hits are listed within this table. Incidences are given for each animal (column 1), as well as for each compound (column 2 and 3). Compound incidences were based on averages across all animals (usually three) for the 5 and 7 day highest dose replicate. A compound incidence was counted if the severity average for the replicate was greater than 0, with the definition of the severity grades as follows: normal=0, minimal=1, mild=2, moderate=3, and marked=4. For comparison purposes, control replicates were formed with three animals per replicate (replicate formation was compound study date-based). This resulted in a total of 112 mock treated liver control triplicates used for the control analysis. The same average severity grade rule was used for the control calculation (columns 4-6). The table shows the number of total treatments (N) for each animal, compound (drug), and control (C) examined for both liver and kidney.

As shown in Table 4, the most common compound-induced finding in liver was hepatocyte enlargement, with 98 of 451 compounds (21.7%) causing the pathology.

TABLE 4
FIXED HISTOPATHOLOGICAL VOCABULARY/LIVER, KIDNEY, SPLEEN, HEART, and INTESTINE

| | Animals | Drugs | | Animals | Controls | |
|---|---|---|---|---|---|---|
| LIVER | (N = 1,431) | (N = 461) | | (N = 349) | (N = 112) | |
| HEPATOCYTE ENLARGEMENT | 262 | 21.7% | 98 | 9 | 4.5% | 5 |
| INCREASED EOSINOPHILIC GRANULAR CYTOPLASM | 246 | 21.3% | 96 | 9 | 4.5% | 5 |
| FATTY CHANGE | 140 | 17.7% | 80 | 30 | 19.8% | 22 |
| LEUKOCYTOSIS | 63 | 10.0% | 45 | 14 | 9.9% | 11 |
| APOPTOSIS | 79 | 7.3% | 33 | 0 | 0.0% | 0 |
| NECROSIS | 43 | 6.0% | 27 | 6 | 5.4% | 6 |
| SUBCAPSULAR NECROSIS | 21 | 3.5% | 16 | 2 | 1.8% | 2 |
| INCREASED CELLULAR GLYCOGEN | 23 | 1.8% | 8 | 7 | 0.0% | 0 |
| PORTAL LEUKOCYTOSIS | 10 | 1.8% | 8 | 0 | 1.8% | 2 |
| BILE DUCT HYPERPLASMA | 18 | 1.6% | 7 | 0 | 0.0% | 0 |
| CENTROLOBULAR HYDROPIC CHANGE | 19 | 1.6% | 7 | 0 | 0.0% | 0 |
| BILIARY LEUKOCYTE INFILTRATION | 12 | 1.1% | 5 | 0 | 0.0% | 0 |
| HEPATOCYTE PALLOR | 11 | 1.1% | 5 | 0 | 0.0% | 0 |
| PERITONITIS | 10 | 1.1% | 5 | 0 | 0.0% | 0 |
| CONGESTION | 4 | 0.7% | 3 | 1 | 0.0% | 0 |
| FRESH HEMORRHAGE | 6 | 0.4% | 2 | 0 | 1.8% | 2 |
| INCREASED MITOTIC NUCLEI | 3 | 0.4% | 2 | 2 | 0.0% | 0 |
| BILE DUCT NECROSIS | 2 | 0.2% | 1 | 0 | 0.0% | 0 |
| CAPSULE FIBROSIS | 5 | 0.2% | 1 | 2 | 0.9% | 1 |
| FIBROSIS | 1 | 0.2% | 1 | 1 | 1.8% | 2 |
| MINERALIZATION | 2 | 0.2% | 1 | 0 | 0.0% | 0 |
| ACUTE INFLAMINATION | 0 | 0.0% | 0 | 1 | 0.9% | 1 |
| AUTOLYSIS | 3 | 0.0% | 0 | 1 | 0.0% | 0 |
| CAPSULE ADHESION | 0 | 0.0% | 0 | 0 | 0.9% | 1 |
| HYDROPIC CHANGE | 1 | 0.0% | 0 | 0 | 0.0% | 0 |
| LEUKOCYTE INFILTRATION | 0 | 0.0% | 0 | 0 | 0.9% | 1 |
| MALIGNANT LYMPHOMA | 0 | 0.0% | 0 | 1 | 0.9% | 1 |
| Number of drugs with no findings | | 52.1% | 235 | | 58.9% | 66 |
| | Animals | Drugs | | Animals | Controls | |
| KIDNEY | (N = 1,279) | (N = 126) | | (N = 84) | (N = 29) | |
| CORTICAL TUBUBLAR DILATION | 13 | 5.6% | 7 | 0 | 0.0% | 0 |
| PELVIS DILATION | 6 | 4.0% | 5 | 2 | 6.9% | 2 |
| CORTICAL TUBULAR VACUOLATION | 8 | 4.0% | 5 | 0 | 0.0% | 0 |
| TUBULAR REGENERATION | 7 | 3.2% | 4 | 1 | 3.4% | 1 |
| PROXIMAL TUBULAR NECROSIS | 11 | 3.2% | 4 | 0 | 0.0% | 0 |
| CORTEX CYST(S) | 3 | 2.4% | 3 | 2 | 6.9% | 2 |
| CORTICAL TUBULAR CAST(S) | 5 | 2.4% | 3 | 0 | 0.0% | 0 |
| LEUKOCYTOSIS | 4 | 2.4% | 3 | 2 | 3.4% | 1 |
| CORTEX FIBROSIS | 2 | 1.6% | 2 | 0 | 0.0% | 0 |
| REGENERATION | 2 | 1.6% | 2 | 0 | 0.0% | 0 |
| CYST | 3 | 1.6% | 2 | 0 | 0.0% | 0 |
| CORTICAL TUBULAR CALCULI | 3 | 0.8% | 1 | 0 | 0.0% | 0 |
| HYDRONEPHROSIS | 1 | 0.8% | 1 | 0 | 0.0% | 0 |
| TUBULE DILATION PAPILLA | 1 | 0.8% | 1 | 0 | 0.0% | 0 |
| PELVIS UROTHELIAL HYPERPLASIA | 1 | 0.8% | 1 | 0 | 0.0% | 0 |
| SUBACUTE VASCULITIS | 1 | 0.8% | 1 | 0 | 0.0% | 0 |
| FIBROSIS | 1 | 0.8% | 1 | 0 | 0.0% | 0 |
| CASTS, PROTEIN | 0 | 0.0% | 0 | 1 | 0.0% | 0 |
| Number of drugs with no findings | | 71.4% | 90 | | 79.3% | 23 |

Many xenobiotics were found induce cytochrome P450 enzyme expression, which induces expansion of the endoplasmic reticulum and hepatocyte enlargement. Hepatocellular hypertrophy was also found spontaneously in 4.5% of the vehicle control “treatments.” The most common pathological finding in kidney was cortical tubular dilation, occurring in 7 of 126 (5.6%) compounds that were examined. This pathology was not found in any vehicle control animals.

### Example 3

### Correlative Use of Chemical Genomic Database

A. Chemogenomic Effects of Anti-Cancer Drugs

Many anti-cancer drugs are known to cause toxicity to the bone marrow hematopoietic progenitor cells by directly damaging DNA or inhibiting its synthesis in cells of this highly proliferative tissue. Anti-cancer drugs known to deplete bone marrow include carmustine, thioguanine, and methotrexate, which block cellular proliferation by different mechanisms. Carmustine is a nitrosourea-class free oxygen radical generator and DNA alkylator, methotrexate is a dihydrofolate reductase inhibitor that interferes with the synthesis of purine nucleotides and dTMP, and thioguanine is a thiopurine compound that acts by multiple mechanisms including direct incorporation into DNA, inhibition of DNA synthesis, and inhibition of purine nucleotide biosynthesis.

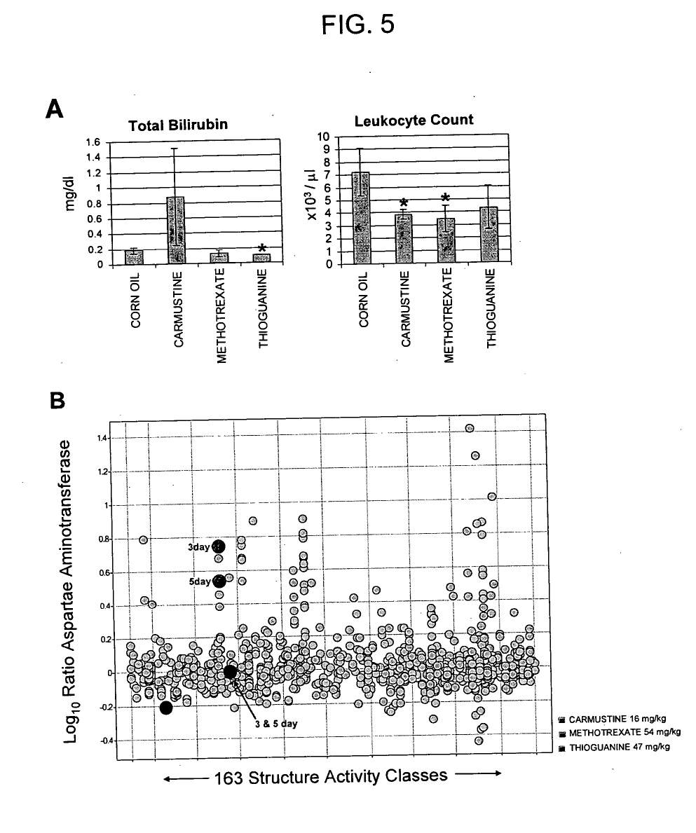

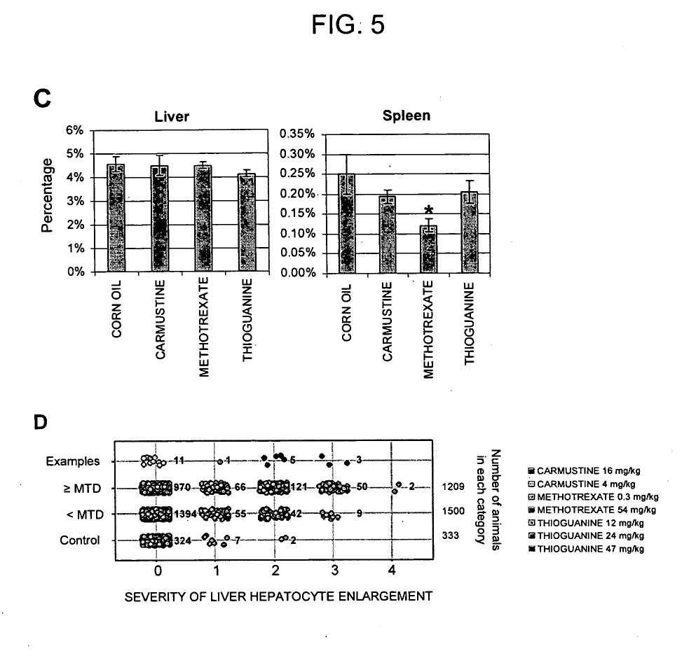

Several different clinical endpoints were affected by these three anti-cancer drugs, based on several clinical assays, hematology assays, organ weights, and histopathology observations as displayed in FIG. 5 (A-D). FIG. 5A shows total bilirubin levels (mg/dl) and leukocyte counts (1000/μl) for carmustine, methotrexate, and thioguanine (y-axis). Data for quadruplicate animals is shown after 3 days of dosing at the MTD; asterisks indicate averages that are statistically different from the controls with a p-value of <0.01. FIG. 5B displays the log₁₀ ratios for aspartate aminotransferase measured in serum across a total of 891 liver treatments (only 3, 5, and 7 day treatments) for a total of 322 different compounds. The x-axis separates the compounds by structure activity classes (total of 163 classes). The doses were as follows: carmustine (3 and 5 day at 16 mg/kg), methotrexate (3 day at 54 mg/kg), and thioguanine (3 and 5 day at 47 mg/kg) treatments are highlighted in red, green, and blue respectively. FIG. 5C depicts the average organ weights for liver and spleen relative to the body weight. Data presented are averages of three animals for each compound treatment. Asterisks indicate averages that are statistically different from the controls with a p-value of <0.01. FIG. 5D depicts the histopathology findings of liver hepatocyte enlargement in terms of severity scores observed for a total of 2,709 experimental animals (2,653 at day 5 and 56 at day 7) and 333 control animals (321 at day 5 and 12 at day 7). The data are grouped according to whether the dose administered in each treatment is >=MTD, <MTD or is a vehicle dosed animal (controls). The number of animals at each severity level was tallied next to that group of colored circles in the figure. Compound incidences (severity scores) were based on averages across all animals (usually three) for the 5 and 7 day highest dose replicate. A compound incidence was counted if the severity average for the replicate was greater than 0, with the definition of the severity grades as follows: normal=0, minimal=1, mild=2, moderate=3, and marked=4. The 5 day carmustine, methotrexate, and thioguanine drug treatments at both MTD and therapeutic levels (FED) are used as examples to demonstrate that the changes caused by these three anti-neoplastic drugs are more frequent than found in many other drugs.

As summarized in FIG. 5A, all three drugs deplete leukocytes, consistent with their anti-proliferative mode of action, as do 26 other drugs of approximately 600 tested (Table 2), but only carmustine (day 3) increased bilirubin levels (FIG. 5A). Bilirubin increases are generally associated with cholestasis, which is the term used to describe impaired hepatic bile duct flow. Average increases of bilirubin of more than 4 fold relative to controls after three days of treatment are relatively rare, with only 11 in ˜600 other compounds having this property. In addition, of the three compounds, only carmustine significantly elevated the serum level of the hepatotoxicity marker Aspartate Aminotransferase (AST; see FIG. 5B). Only 17 other drugs of ˜600 elevate AST to the extent that carmustine does (data not shown). In terms of drug-induced organ weight changes, methotrexate, and to a lesser extent carmustine and thioguanine, decreased the relative spleen weight as shown in FIG. 5C, consistent with impaired blood cell proliferation and the resultant depletion of blood reservoirs in the spleen. In contrast, none of the three compounds affected relative liver weight (FIG. 5C). Histopathologically, hepatocyte enlargement occurred in several of the animals treated with each of the three compounds (FIG. 5D). However, unlike thioguanine and methotrexate, only carmustine was found to produce histological evidence of mild bile duct hyperplasia which is consistent with its effect on bilirubin levels. Taken together, it appears that based on traditional clinical endpoint measurements all three drugs cause bone marrow toxicity and some hepatotoxicity, with carmustine being a more severe hepatotoxicant, causing overt bile duct hyperplasia, and large AST increases.

The ability to benchmark changes relative to many other compounds allows one to make rapid conclusions about the significance of an event; for example, using the database with data for 600 compounds it may be rapidly concluded that it is unusual for a strong marrow toxicant to also be a strong bile duct toxicant.

B. Association of Expression of Single Genes with the Anti-Proliferative Action of the Anti-Cancer Compounds

A correlation analysis was performed to determine which liver gene expression changes are most closely associated with leukocyte depletion, in that all three of the anti-cancer drugs depleted this cell type from peripheral blood as described above. There were 877 liver drug-dose-time combinations (consisting of triplicate animals) in the liver dataset of the database where leukocyte counts were measured in the blood of the same animals whose livers were subjected to microarray analysis. A Pearson's correlation was computed between these leukocyte counts (expressed as log₁₀ ratios to controls) and each of the 8,565 probes measured in liver across the 877 treatments. Since leukocyte depletion is a blood compartment-specific event, the correlation data of liver probes to leukocyte count should be interpreted in the context of blood cell expression data. For this purpose the average steady state expression levels for all 8,565 probes in normal blood cells were sorted according to their absolute normalized fluorescence intensity in whole blood from highest to lowest expression.

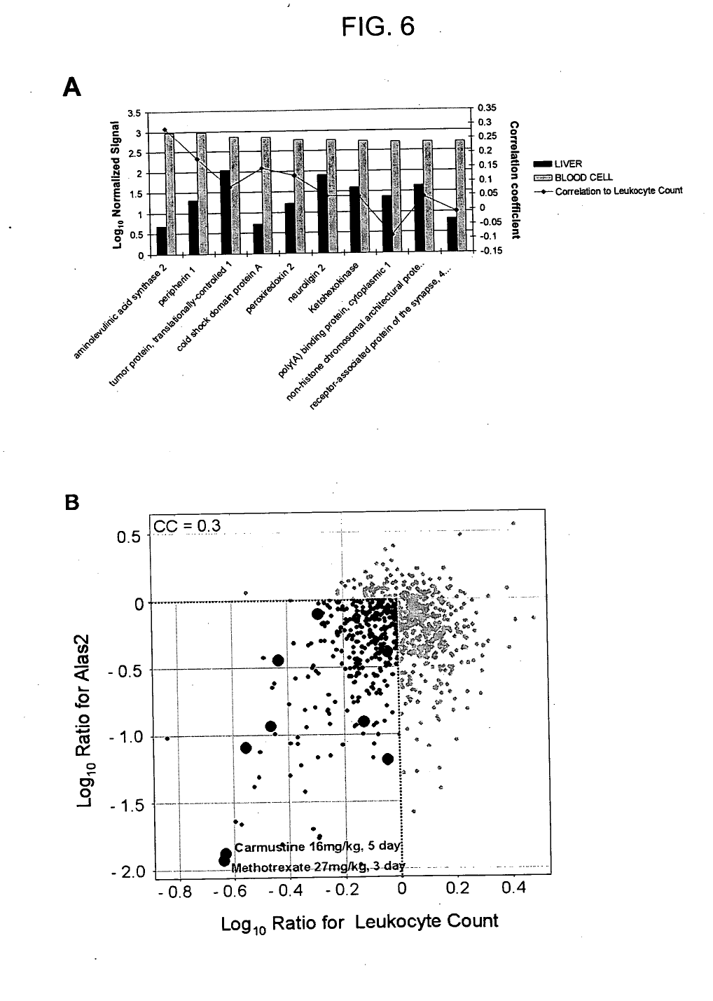

FIG. 6A shows log₁₀ signal intensities in whole blood (grey bars) and liver (black bars) are displayed for the 10 RNAs with highest expression in normal blood cells. The average steady state expression is shown for those probes and is calculated from vehicle-treated controls. Overlaying these steady state expression levels is a red line that plots the correlation of each probe to leukocyte count (right y-axis) based on their drug-treated liver expression pattern (across a total of 877 liver 5 day treatments).

The 10 probes with the highest expression levels in whole blood as measured on microarrays, with their lower expression levels in normal liver shown for comparison and overlaid with the aforementioned Pearson's correlation data. The positive correlation of these transcripts with leukocyte counts suggests that these transcripts are not only highly expressed in blood (indeed, they are blood selective, having lower expression in 12 other tissues and primary hepatocytes, data not shown), but that they are also depleted along with leukocytes and presumably other blood cells by these drug treatments. Aminolevulinate synthase 2 (Alas2) (GenBank NM_{—}013197) was observed to have the highest expression level in blood and one of the highest correlations in liver (5^{th} highest correlation among all 8,565 probes) to the leukocyte count. Alas2 was identified as a reticulocyte-specific gene induced during erythropoiesis and essential for this function, since its absence (by mutation) can cause X-linked sideroblastic anemia (Bishop, D. F., A. S. Henderson, and K. H. Astrin, “Human delta-aminolevulinate synthase: assignment of the housekeeping gene to 3p21 and the erythroid-specific gene to the X chromosome,: Genomics 7: 207-214 (1990)). Its gene product is responsible for catalyzing the essential, committed step of heme biosynthesis, and even Alas1, the ubiquitous isoform of the enzyme, cannot compensate for loss of Alas2 expression (Sadlon, T. J., T. Dell'Oso, K. H. Surinya, and B. K. May, “Regulation of erythroid 5-aminolevulinate synthase expression during erythropoiesis. 31(10): 1153-1167 (1999)).

FIG. 6B plots the log ratios for Alas2 in liver versus the leukocyte count across all 877 liver treatments, a scatter plot with an overall correlation of 0.3 (or 0.6 for liver treatments with significant leukocyte decrease and down regulated Alas2 expression). The chart in FIG. 6B shows Alas2 logo expression ratio (y-axis) versus leukocyte count log₁₀ ratios (x-axis) across the averages of the liver treatments. Highlighted in red are the values for the 3 and 5 day treatments of carmustine, thioguanine, and methotrexate anti-cancer drug treatments. Only treatments with significant (p-value<0.05) Alas2 expression are used for the generation of this graph. The correlation coefficient across the 877 different treatments is 0.3 as is shown in the upper left corner. The low correlating experiments with slightly up regulated Alas2 and/or leukocyte increases are shaded gray.

Analysis of the expression of Alas2 in the context of the entire database reveals that this gene is depleted from multiple tissues (i.e. spleen, bone marrow, heart, and liver tissues) by a number of compounds, most of which have anti-neoplastic therapeutic activities that block cell proliferation. As shown in Table 5, the most profound suppression of Alas2 in the entire database was seen in spleen, where a thioguanine treatment (24 mg/kg daily for 5 days) lowers the expression level of Alas2 a log₁₀ ratio of −2.77, or nearly 600-fold relative to vehicle treated controls.

TABLE 5

| | | | | | | Log10 Ratio to Control | |
|---|---|---|---|---|---|---|---|
| | | Dose | Time | Dose | | Alas2 | Leukocyte |
| Drug | Structure_Activity_Class (Therapeutic_Class*) | (mg/kg) | days | Level | Tissue | Expression | Count |
| 1 THIOGUANINE | DNA-Polymerase Inhibitor, thiopurine base (AN) | 24 | 5 | MTD | SP | −2.77 | −0.15 |
| 2 DOXORUBICIN | DNA intercalator, anthracycline (AN) | 3 | 5 | MTD | SP | −2.62 | −0.58 |
| 3 VINCRISTINE | Tubulin binder, vinca (AN) | 0.05 | 5 | NA | HE | −2.37 | −0.19 |
| 4 METHOTREXATE | Antifolate, dihydrofolate reductase inhibitor (AN, IS) | 27 | 3 | MTD | SP | −2.31 | −0.64 |
| 5 ETOPOSIDE | DNA topoisomerase II inhibitor (AN) | 188 | 5 | MTD | BM | −2.31 | −0.30 |
| 6 DAUNORUBICIN | DNA intercalator, anthracycline (AN) | 3.25 | 5 | MTD | HE | −2.25 | −0.85 |
| 7 MITOXANTRONE | DNA intercalator (AN) | 2 | 5 | MTD | HE | −2.23 | −0.98 |
| 8 VINCRISTINE | Tubulin binder, vinca (AN) | 0.05 | 5 | NA | BM | −2.19 | −0.19 |
| 9 HYDROXYUREA | Ribonucleoside-PP reductase inhibitor (AN) | 400 | 5 | MTD | SP | −2.16 | −0.35 |
| 10 VINBLASTINE | Tubulin binder, vinca (AN) | 0.3 | 3 | MTD | HE | −2.14 | −0.32 |
| 11 IFOSFAMIDE | DNA-alkylator, nitrogen mustard (AN) | 143 | 5 | NA | SP | −2.13 | −0.51 |
| 12 THIOGUANINE | DNA-Polymerase inhibitor, thiopurine base (AN) | 12 | 3 | NA | SP | −2.12 | −0.56 |
| 13 ETOPOSIDE | DNA topoisomerase II inhibitor (AN) | 188 | 3 | MTD | SP | −2.12 | 0.04 |
| 14 MITOXANTRONE | DNA intercalator (AN) | 2 | 3 | MTD | HE | −2.10 | −0.88 |
| 15 IFOSFAMIDE | DNA-alkylator, nitrogen mustard (AN) | 143 | 3 | NA | SP | −2.10 | −0.60 |
| 16 ETOPOSIDE | DNA topoisomerase II inhibitor (AN) | 188 | 3 | MTD | BM | −2.08 | 0.04 |
| 17 VINBLASTINE | Tubulin binder, vinca (AN) | 0.3 | 5 | MTD | HE | −2.06 | −0.43 |
| 18 EPIRUBICIN | DNA intercalator, anthracycline (AN) | 2.7 | 5 | MTD | HE | −2.05 | −0.93 |
| 19 ETOPOSIDE | DNA topoisomerase II inhibitor (AN) | 100 | 3 | NA | SP | −2.03 | −0.34 |
| 20 THIOGUANINE | DNA-Polymerase inhibitor, thiopurine base (AN) | 24 | 5 | MTD | LI | −1.97 | −0.15 |
| 21 IFOSFAMIDE | DNA-alkylator, nitrogen mustard (AN) | 143 | 5 | NA | HE | −1.93 | −0.51 |
| 22 CARMUSTINE | DNA damaging, nitrosourea (AN) | 16 | 5 | MTD | LI | −1.92 | −0.64 |
| 23 DOXORUBICIN | DNA intercalator, anthracycline (AN) | 3 | 5 | MTD | HE | −1.91 | −0.58 |
| 24 DOXORUSICIN | DNA intercalator, anthracycline (AN) | 3 | 3 | MTD | SP | −1.90 | −0.53 |
| 25 VINBLASTINE | Tubulin binder, vinca (AN) | 0.3 | 5 | MTD | LI | −1.89 | −0.43 |
| 26 METHOTREXATE | Antifolate, dihydrofolate reductase inhibitor (AN, IS) | 27 | 3 | MTD | LI | −1.87 | −0.64 |
| 27 LEFLUNOMIDE | Inhibits pyrimidine/purine metabolism (ADMA) | 60 | 5 | MTD | SP | −1.87 | −0.45 |
| 28 THIOGUANINE | DNA-Polymerase inhibitor, thiopurine base (AN) | 24 | 5 | MTD | BM | −1.83 | −0.15 |
| 29 VINBLASTINE | Tubulin binder, vinca (AN) | 0.3 | 3 | MTD | LI | −1.77 | −0.32 |
| 30 ETOPOSIDE | DNA topoisomerase II inhibitor (AN) | 188 | 5 | MTD | LI | −1.77 | −0.30 |

*AN = Antineoplastics;
IS = immunosuppressants;
ADMA = Antirheumatic Disease Modifying Agents

C. Alas2 and Reticulocyte Depletion

Because of the essential role of Alas2 in erythrocyte heme production, it is likely that the level of its transcript is essentially a surrogate marker for reticulocytes, which, like the leukocytes, must be unable to properly develop in the bone marrow due to the activity of the anti-neoplastic agents. To confirm this, reticulocyte staining and counting was followed by microarray analysis after a three day repeat dose study of methotrexate, thioguanine, and carmustine.

The reticulocyte staining and counting protocol used was based on examination of microscopic blood smears stained with the rRNA precipitating cationic dye “New Methylene Blue” (Sigma Chemicals, St. Louis, Mo.). Three drops of EDTA-treated whole blood were mixed with two drops of reticulocyte stain (New Methylene Blue). The sample was incubated at room temperature for 10 min before a thin smear was spread on a microscope slide. After 5 minutes, the reticulocytes were counted under an oil-immersion using a Miller Disc. This method requires counting 1000 RBC and converting this count to percentage of reticulocytes per 100 RBC. The absolute count was then calculated using the percentage of reticulocytes times the total RBC per micro liter.

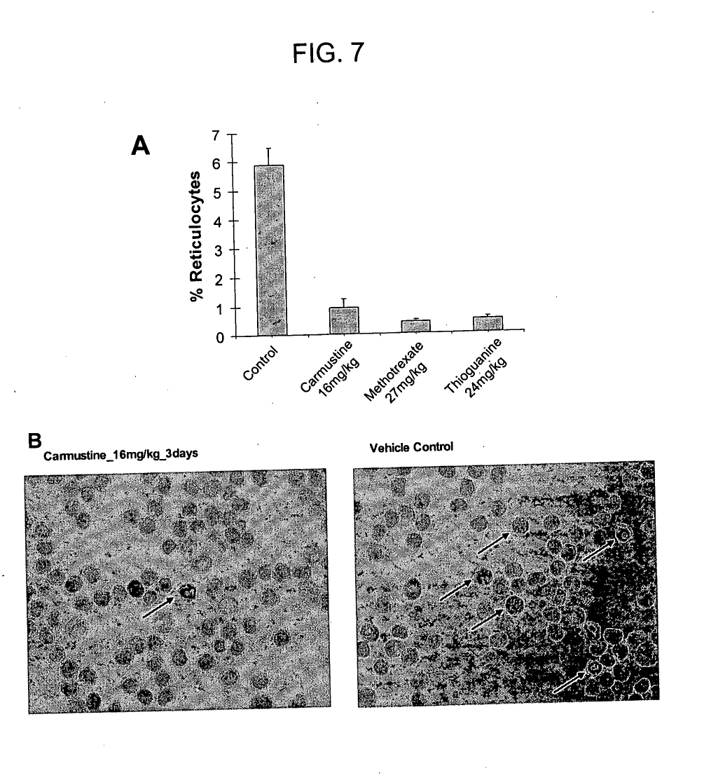

As shown in FIG. 7A, substantial decreases in reticulocyte counts were observed for these drug treatments, with methotrexate having the most pronounced effect (15-fold), followed by thioguanine (12-fold) and carmustine (6-fold). An example of a Methylene Blue stained peripheral blood smear from a carmustine-treated rat is shown to illustrate the reticulocyte depletion (FIG. 7B). To complete the study, the treated samples were also analyzed at the gene expression level to monitor mRNA levels in liver in the same animals. The livers of these compound-treated rats were subjected to microarray analysis. As shown earlier with other anti-cancer treatments, Alas2 transcripts were reduced on average about 20-fold as compared to vehicle treated controls (data not shown), in agreement with the decreased amounts of reticulocytes (and leukocytes; FIG. 7A) within these samples.

Therefore, Alas2 can serve as a biomarker for depletion of reticulocytes when its mRNAs is strongly repressed. Alas2 has been previously described as reticulocyte specific (Bishop, D. F., A. S. Henderson, and K. H. Astrin, “Human delta-aminolevulinate synthase: assignment of the housekeeping gene to 3p21 and the erythroid-specific gene to the X chromosome,: Genomics 7: 207-214 (1990)), but this is the first description of an association of this gene to its functional location within the blood compartment, identified using a contextual chemogenomics data source, i.e. clinical data combined with expression data. Furthermore, we suggest that this biomarker might be useful as an investigative tool to study the effect of chemical treatments on hematopoiesis. In addition, analyzing the clinical pathology data within the same animal across different tissues revealed that blood and bone marrow-specific effects on blood-selective markers are detectable in liver and several other non-hematopoietic tissues (see Table 4). This finding indicates that global gene expression analysis from one tissue (e.g. liver), may be used to detect and/or monitor compound effects occurring in other tissues and/or other cellular compartments (e.g. blood).

D. Similarities in Gene Expression: Carmustine, Methotrexate and Thioguanine Perturb Cell Cycle and Blood-Specific Genes

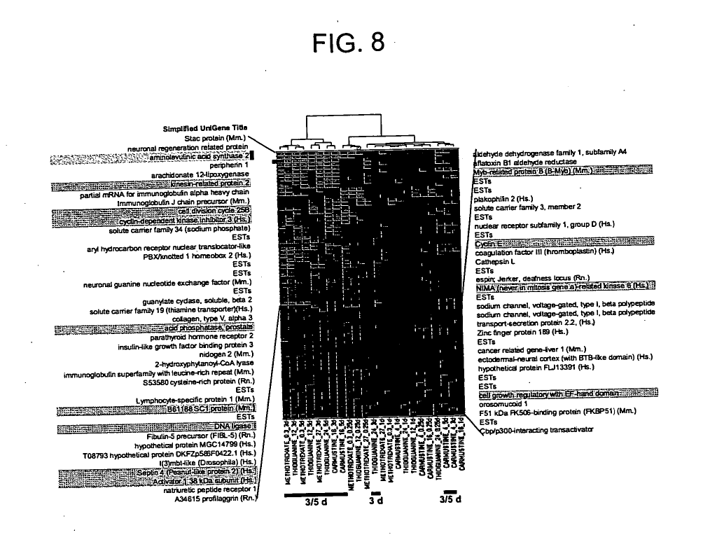

To more thoroughly examine the liver gene expression changes that are shared among the three anti-cancer drugs, hierarchical clustering was performed on expression data for selected genes among the 23 individual dose-time combinations available for carrnustine, methotrexate, and thioguanine. The clustering shown in FIG. 8 is based on the 73 genes (of the ˜8500 that were measured) that were significantly (p<0.05) perturbed in at least 35% (i.e. in at least 8 of 23) of these drug-dose-time combinations in liver. The clustering was carried out using correlation as the similarity metric (unweighted average method). The continuous color intensity in the figure was scaled so that log₁₀ ratios of +0.6 (induced genes) correspond to bright red, and log₁₀ ratios of −0.6 (repressed genes) correspond to bright green, and black denotes a log₁₀ ratio of 0. The two lists on either side are the gene names. Log₁₀ ratios were set to 0 if they did not achieve a t-test significance of p<0.01 comparing the biological triplicates to control expression levels. Genes shaded in light grey are genes significantly associated with the GO term cell cycle (p<0.004), using the hypergeometric analysis of the distribution of GO for 6,327 different genes with GO assignments present on RU1 arrays. The blood selective gene Alas2 is shaded with light blue. Of the different drug-dose combinations, the later time points (3 and 5 day) form a separate cluster away from the earlier time points. Applying this gene enrichment approach led to the identification of a subset (12 genes) of the 73 genes that have a significant enrichment (p<0.004) for the Gene Ontology (GO) terms associated with the cell cycle (highlighted in light gray in FIG. 8).

Gene Ontology (GO) annotations to help interpret the gene expression changes induced by the compound of interest. Gene Ontology (GO) analysis using the GO Data Visualization Tool. The GO tool takes a list of genes as an input and generates Gene Ontology annotations that describe a gene product in terms of three hierarchies, (1) Molecular Function (MF, the biochemical activity of the protein, e.g. Kinase), (2) Biological Process (BP, biological role of the protein in an organism, e.g. Cell cycle control), and (3) Cellular Component (CC, the place in a cell where the protein is active, e.g. Nucleus). The p-value is the hypergeometric probability of seeing a GO term for the list of genes by chance, evaluated by comparing with the distribution of the words associated with all of the genes on the chip.

Furthermore, Alas2, whose expression level tracks with leukocyte and reticulocyte levels (see above), is among these 73 genes and is highlighted near the top of the heat map in FIG. 8 (light blue shading). The observed depletion of the Alas2 transcript is greater at later time points than earlier time points for each of the 23 drug-dose combinations.

To summarize, the gene expression data when correlated the clinical bioassay data highlights those critical genes regulating the cell cycle that are perturbed by these drugs, and also those biomarkers of blood cells that decrease in liver tissue.

E. Differences in Gene Expression: Carmustine Treatments Perturb Genes Associated with Bile Duct Hyperplasia.

Several different clinical endpoints are collected for each compound treatment; one is the histopathology observation based on the standard histopathology vocabulary and grading system which is used to assess the effect of a compound on various tissues. A small number of compounds were identified in the database that induce bile duct hyperplasia (BDH), among them the well-known inducers 1-naphthyl isothiocyanate (ANIT), lomustine, 4,4′-diaminodiphenylmethane (methylene dianiline), and methapyrilene. Interestingly, as mentioned earlier, carmustine was likewise a mild inducer of BDH at high doses and later time points, whereas thioguanine and methotrexate were not. Bile duct hyperplasia occurs when the epithelial cells that line the bile ducts (cells known as cholangiocytes) proliferate in response to bile acids and xenobiotics, including toxicants. Hierarchical clustering was used to qualitatively explore the genes that are associated with the histopathology and development of BDH and several other markers of liver injury, and to use this information to examine the differences between carmustine and the other two anti-neoplastic drugs.

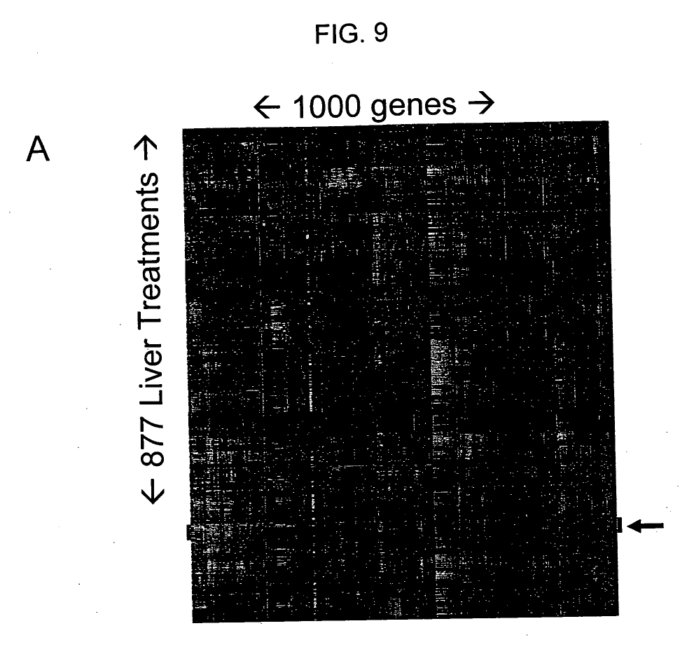

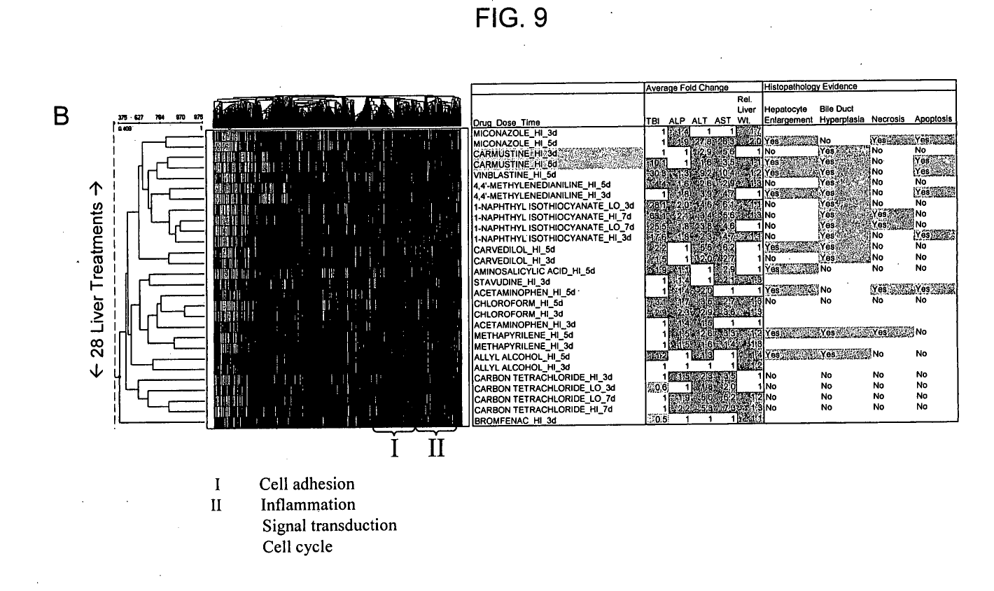

The 1000 most perturbed genes (by standard deviation of their log ratio across 877 liver treatments) among all 877 liver treatments of 3, 5 or 7 days duration were hierarchically clustered. The resultant clustering, depicted in FIG. 9A, was performed using the correlation similarity metric (unweighted average method). The continuous color intensity is scaled so that log₁₀ ratios of +0.5 (induced genes) correspond to bright red, and log₁₀ ratios of −0.5 (repressed genes) correspond to bright green, and black denotes a log₁₀ ratio of 0. The subcluster containing the two high dose carmustine treatments is highlighted with green bars. The high dose carmustine treatments were found in a cluster with a number of other hepatotoxicants, with an overall correlation coefficient of 0.408. This subcluster of 28 treatments (FIG. 9B) contained several drugs that caused clinically measured increases in ALP, ALT, and AST, and many which also inflicted histopathologically evident BDH. The carmustine subcluster has an overall correlation coefficient of 0.408. The tables depicted to the right of of the clustering (in FIG. 9B) summarize individual observed clinical outcomes, i.e. the average fold changes for different liver enzyme measurements, bilirubin levels, and relative liver weight, and a summary of the histopathological findings. Highly correlating genes were grouped according to their annotation, i.e. (I) genes encoding cell adhesion molecules, and (II) genes encoding inflammation/cell cycle/signal transduction specific genes.

Algorithms may be used that generate linear classifiers based on classification hypotheses used to query the database. Useful algorithms include those based respectively on Support Vector Machines (SVM), Logistic regression (LR) and Minimax Probability Machine (MPM). Such algorithms have been described in detail elsewhere (See e.g., El Ghaoui et al., “Robust classifiers with interval data” Report # UCB/CSD-03-1279. Computer Science Division (EECS), University of California, Berkeley, Calif. (2003); Brown, M. P., W. N. Grundy, D. Lin, N. Cristianini, C. W. Sugnet, T. S. Furey, M. Ares, Jr., and D. Haussler, “Knowledge-based analysis of microarray gene expression data by using support vector machines,” Proc Natl Acad Sci U S A 97: 262-267 (2000)).

Using Support Vector Machine (SVM) technology a drug signature was generated that classifies those drugs that cause BDH from those that do not. A number of the genes that compose the resulting drug signature were found in the clustering of the compound treatments, and are indicated by I (cell adhesion, extracellular matrix, and morphology) and II (inflammation, cell cycle, and signal transduction) at the bottom of FIG. 9B. Two high impact genes in the BDH signature that are part of this cluster include the cell adhesion molecule tenascin C (AA892824) and the inflammation-specific gene lipocalin 2 (X13295) (data not shown, but indicated by I and II in FIG. 9B). Carmustine's effect on the majority of the genes is quite different than thioguanine or methotrexate as each of these drugs clusters away from carmustine.

To summarize, the analysis of gene expression profiles in the above described chemogenomic database confirms and elaborates on the differences between the three anti-cancer drugs carmustine, thioguanine, and methotrexate, which correlate with differences evident at the histopathological level. A simple unsupervised 2-D clustering identifies the association between carmustine and several other strong hepatotoxicants and provided some molecular detail as to the cellular processes that they perturb.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

1. A method for facilitating exploration of biological and chemical data, comprising:
a) providing a database comprising a plurality of standard gene expression profiles, each profile comprising a representation of the expression level of a plurality of genes in a cell exposed to a standard compound and a representation of the standard compound;
b) displaying a selected gene expression profile;
c) displaying correlation information related to said gene expression profile to facilitate generation of a hypothesis; and
d) displaying relevant product information to facilitate testing said hypothesis.

2. The method of claim 1, wherein said correlation information is selected from the group consisting: identification of a profile similar to said gene expression profile, identification of a compound that produces a similar profile, identification of a gene modulated in said profile, identification of a disease or disorder in which a plurality of the same genes are modulated in a similar fashion, identification of compounds having similar physical and chemical properties as the compound used to generate the profile, identification of compounds having similar shapes, identification of compounds having similar biological activities, identification of a gene or protein having sequence similarity to a selected gene or protein, identification of a gene or protein having a similar known function or activity, identification of a gene or protein subject to modulation or control by the same compound, identification of a gene or protein that belongs to the same metabolic or signal pathway, and identification of a gene or protein belonging to similar metabolic or signal pathways.

3. The method of claim 1, wherein said relevant product information is selected from the group consisting of: information regarding a bioassay reagent useful for measuring activity of an identified enzyme, information regarding a compound useful as a positive control, information regarding a compound useful as a negative control, information regarding a kit for purifying an identified protein, information regarding antibodies for determining and/or isolating substances, information regarding a compound similar to the test compound useful for further study, additional data regarding gene or protein function and/or relationships, sequence data from other species, information regarding metabolic and/or signal pathways to which the gene or protein belong, information regarding a DNA microarray useful for determining expression of the gene and/or related genes, and information and analysis regarding features of a compound that are likely to be responsible for the observed activity.

4. The method of claim 3, wherein said product information further comprises a hyperlink that facilitates direct purchase of said product.

5. The method of claim 1, wherein said database further comprises drug signatures for a plurality of compounds, wherein each said drug signature comprises a representation of the physical and chemical characteristics of each compound, data regarding the effect of each compound on the transcription of a plurality of genes, and data regarding the effect of each compound on a plurality of proteins.

6. The method of claim 1, wherein said gene expression profile is selected on the basis of its similarity to an experimental expression profile provided by the user.

7. A system for facilitating exploration of biological and chemical data, comprising:
a database comprising a plurality of standard gene expression profiles, each profile comprising a representation of the expression level of a plurality of genes in a cell exposed to a standard compound and a representation of the standard compound;
input means for accepting data and user selections;
selection means for selecting a gene expression profile;
correlation selection means for identifying correlation information related to said gene expression profile;
product information selection means for selecting information regarding relevant products related to said gene expression profile; and
display means for displaying information regarding said gene expression profile.

8. The system of claim 10, wherein said database further comprises drug signatures for a plurality of compounds, wherein each said drug signature comprises a representation of the physical and chemical characteristics of each compound, data regarding the effect of each compound on the transcription of a plurality of genes, and data regarding the effect of each compound on a plurality of proteins.

9. A system for facilitating exploration of biological and chemical data, comprising:
a database comprising drug signatures for a plurality of compounds, wherein each said drug signature comprises a representation of the physical and chemical characteristics of each compound, data regarding the effect of each compound on the transcription of a plurality of genes, and data regarding the effect of each compound on a plurality of proteins;
input means for accepting data and user selections;
selection means for selecting a gene expression profile;
correlation selection means for identifying correlation information related to said gene expression profile;
product information selection means for selecting information regarding relevant products related to said gene expression profile; and
display means for displaying information regarding said gene expression profile.
